(19) United States
(12) Patent Application Publication
(10) Pub. No.: US 2022/0409510 A1
(43) Pub. Date: Dec. 29, 2022

(54) Polypeptides And Methods For Improving Skin Conditions

(71) Applicant: Interk Peptide Therapeutics Limited, New South Wales (AU)

(72) Inventor: Michael Agrez, New South Wales (AU)

(73) Assignee: Interk Peptide Therapeutics Limited, New South Wales (AU)

(21) Appl. No.: 17/298,083

(22) PCT Filed: Nov. 29, 2019
(86) PCT No.: PCT/AU2019/051313

§ 371 Date: May 28, 2021

(30) Foreign Application Priority Data
Nov. 30, 2018 (AU) 2018904570

(51) Int. Cl. A61K 8/64; A61Q 19/00; A61Q 19/08

(52) U.S. Cl. CPC A61K 8/64; A61Q 19/004; A61Q 19/08

(57) ABSTRACT

The present invention relates to polypeptides, compositions and methods for preventing and/or treating skin conditions including dermal aging and skin conditions associated with UV exposure.

### TECHNICAL FIELD

The present disclosure relates to polypeptides, compositions and methods for preventing and/or treating skin conditions including dermal aging and skin conditions associated with UV exposure.

### BACKGROUND OF INVENTION

The cellular and molecular mechanisms of skin aging, skin damage and skin wound healing are not fully understood.

Skin aging is associated with loss of elasticity, dryness, wrinkles and pigmentation. Ultraviolet (UV) irradiation to the skin and, in particular, exposure to UVB having a wavelength of 290 to 320 nm, is known to cause chronic skin damage that includes premature skin aging and induction of skin cancer. Skin cancer costs the Australian health system more than $700 million each year and the estimated number of new skin cancer cases diagnosed in 2018 is over 138,000.

UVB irradiation has been shown to induce expression of cyclooxygenase-2 (COX-2) with up-regulation of COX-2 playing a functional role in UVB-mediated tumour promotion. Moreover, the cyclic AMP response element binding protein (CREB) is phosphorylated and activated upon UVB treatment and is responsible for UVB-induced COX-2 expression.

The repair of photo-damaged DNA requires large amounts of cellular energy to unwind and remodel its compact chromatin structure to enable access of repair enzymes to the damaged DNA. The main source of energy within a cell is adenosine triphosphate (ATP). Exposure of the skin to UV-radiation puts the skin in a state of energy stress and the production of ATP is reduced, in part due to oxidative damage which inhibits mitochondrial function. The reduction in ATP availability also reduces the efficacy of the immune system, leading to UV induced immunosuppression. In this energy-deprived state following UV exposure the number of effector and memory T cells activated by antigen exposure is low. The immune-suppressive effects of UVB have been recognised for a number of decades.

Ultraviolet exposure, primarily UVB exposure, also leads to increases in reactive oxygen species (ROS) which in turn damage cellular and extra-cellular components such as DNA. Absorption of UV photons drives electrons and energy transfer from cellular photosensitisers, such as porphyrins, bilirubin, melanin, and pterins, to oxygen molecules creating the radical singlet O₂ anion. Consequently, the singlet oxygen anion induces guanine moiety oxidation of DNA followed by structural rearrangement and the formation of 8-hydroxy-2-deoxyguanosine moieties (8-OHdG). 8-OHdG is one of the most important DNA adducts and is used as an indicator of oxidative DNA damage associated with cellular aging and carcinogenesis.

Furthermore UV-irradiation directly affects DNA when DNA absorbs photons from UVB radiation. This results in structural re-arrangement of nucleotides that then lead to defects in the DNA strand. Cyclobutane pyrimidine dimers (CPDs) and pyrimidine (6-4) pyrimidone (6-4 photoproducts, 6-4 PPs) are the major products of UVB-induced DNA damage. Moreover, UVB-induced DNA damage in the form of CPDs can induce mutations in cells (such as epidermal cells) leading to the development of cancer. Reduction of CPDs through application of DNA repair enzymes prevents the risk of UV-induced skin cancer and apoptotic sunburn cells.

Human skin tissue repair is commonly known as wound healing, which is an intricate process where the skin (or another organ-tissue) repairs itself after injury. In normal skin, the epidermis (the outermost layer) and dermis (the inner or deeper layer) exist in a steady-state equilibrium, forming a protective barrier against the external environment. Once the protective barrier is broken or damaged, the process of wound healing is immediately set in motion and may be divided into three or four sequential phases that can overlap and are not mutually exclusive, the phases being: hemostasis, inflammation, proliferation and remodelling.

Within these phases, growth factors cause cell proliferation, thus leading to an integration of dynamic changes that involve soluble mediators, blood cells, the production of the extracellular matrix, and the proliferation of parenchymal cells.

Compromised patients often develop non-healing chronic wounds that have failed to proceed through a timely reparative process to produce anatomic and functional integrity within a period of 3 months. Such wounds present a substantial economic burden to healthcare systems along with significant reductions in quality of life for those affected. The cost in the USA is estimated to be approximately US $20 billion with a UK report suggesting that treatment and care of chronic wounds accounts for 3% of total healthcare expenditure in developed countries.

Agents that can assist with tissue repair and particularly human wound healing are therefore required.

Topical treatments for cosmetic purposes have become a burgeoning field during the past 20 years, especially with polypeptides sequences that have a cosmetic function such as anti-oxidant activity or inhibition of proteases which damage the skins ECM. Of the numerous skincare products that have been developed, many are for merely improving the appearance of human skin and treating signs and symptoms of aging. Other related products act to protect the skin by providing a screen that blocks UV-radiation.

There remains a need for topical compositions that can treat and/or prevent a range of skin conditions including skin photoaging, skin damage and to promote wound healing.

### SUMMARY OF INVENTION

The present inventors have identified that polypeptides including the sequence RSKAKNPLYR, and the corresponding dextro-reverso form of the polypeptide (i.e. rylpnkaksr), when adjacent (e.g. N-terminal of) a polyarginine sequence region, can be used to prevent or treat a range of skin conditions.

Accordingly, the present invention provides an isolated or purified polypeptide comprising the amino acid sequence RSKAKNPLYR (SEQ ID NO: 1), or the dextro-reverso form of the amino acid sequence (i.e. rylpnkaksr—SEQ ID NO: 2), adjacent a polyarginine amino acid sequence region. In some embodiments, the polypeptide is isolated or purified. In some embodiments the polyarginine amino acid sequence region is C-terminal of the amino acid sequence RSKAKNPLYR (SEQ ID NO: 1) or is N-terminal of the dextro-reverso form of the amino acid sequence (i.e. rylpnkaksr—SEQ ID NO: 2).

The polyarginine amino acid sequence region can be any suitable length. However, in some embodiments the polyarginine amino acid sequence region consists of 2 to 20 arginine residues. In some embodiments, the polyarginine amino acid sequence region consists of 4 to 20 arginine residues. In some embodiments, the polyarginine amino acid sequence region consists of 5 to 15 arginine residues. In some embodiments, the polyarginine amino acid sequence region consists of 5 to 12 arginine residues. In some embodiments, the polyarginine amino acid sequence region consists of 5 to 10 arginine residues. In some embodiments, the polyarginine amino acid sequence region consists of 5 to 9 arginine residues. In some embodiments, the polyarginine amino acid sequence region consists of 6 to 8 arginine residues. In some embodiments, the polyarginine amino acid sequence region consists of 7 or 8 arginine residues. In some embodiments, the polyarginine amino acid sequence region consists of 8 arginine residues.

In some embodiments, the polyarginine amino acid sequence region comprises 2, 3, 4, 5, 6, 7, 8, 9, 10, 11, 12, 13, 14, 15, 16, 17, 18, 19 or 20 arginine residues. In some embodiments, the polyarginine amino acid sequence region comprises 8 arginine residues.

Modifications of the C-terminus of the polypeptide of the present invention can change the biological activity of the polypeptides, and more accurately reflect natural polypeptides which typically undergo post-translational modifications. Therefore, in some embodiments, the polypeptide is modified at the C-terminus, preferably amidated at the C-terminus. In some embodiments, the dextro-reverso form of the amino acid sequence is amidated at the C-terminus (i.e. rylpnkaksr-NH₂). In some embodiments, the polyarginine amino acid sequence region is amidated at the C-terminus.

Amino acids are chiral at the alpha carbon adjacent to the carboxyl group, and as such exist as L- and D-isomers. In some embodiments of the invention, the polypeptide comprises L amino acids. In some embodiments of the invention, the polypeptide comprises D amino acids.

As indicated above, the present inventors have identified that the polypeptides of the invention have biological activity, including promoting wound healing. For example. Example 1 shows that the polypeptides IK34720 (SEQ ID NO: 3—RSKAKNPLYRRRRRRRRR) and the dextro-reverso sequence IK236770 (SEQ ID NO: 4—rrrrrrrrrylpnkaksr) promotes wound healing when applied topically, compared to untreated controls.

Accordingly, the present invention provides a polypeptide as described herein for promoting wound healing. In some embodiments, the polypeptide for promoting wound healing comprises (or consists of) RSKAKNPLYRRRRRRRRR (SEQ ID NO: 3). In some embodiments, the polypeptide for promoting wound healing comprises (or consists of) rrrrrrrrrylpnkaksr (SEQ ID NO: 4).

As indicated above, the present inventors have identified that the polypeptides of the invention can treat and/or prevent a skin condition. Accordingly, the present invention provides a polypeptide as described herein for treating or preventing a skin condition. In some embodiments, the polypeptide for treating or preventing a skin condition comprises (or consists of) RSKAKNPLYRRRRRRRRR (SEQ ID NO: 3). In some embodiments, the polypeptide for treating or preventing a skin condition comprises (or consists of) rrrrrrrrrylpnkaksr (SEQ ID NO: 4).

In some embodiments, the skin condition is any one or more of: skin aging; oxidative damage; damage induced by sunlight exposure; ultraviolet radiation induced damage; UVB induced damage; and/or DNA damage.

In some embodiments, one or more of the above skin conditions are characterised by CPD and/or 8-OHdG formation, preferably DNA damage characterised by CPD and/or 8-OHdG formation or ultraviolet induced damage characterised by CPD and/or 8-OHdG formation.

In some embodiments, the polypeptides of the present invention inhibits, in a cell or in the skin of a subject, any one or more of: CREB phosphorylation; 8-OHdG formation; cyclobutane pyrimidine dimer (CPD) formation; matrix metalloproteinase 1 (MMP1) activity; oxidative damage of cellular or extracellular components; and/or DNA damage.

In some embodiments the polypeptides of the present invention increases, in a cell or in the skin of a subject, any one or more of: Adenosine triphosphate (ATP) levels; and/or ultraviolet radiation damage repair.

The polypeptide of the present invention is useful when formulated as a composition. Therefore, the present invention provides a composition for topical use comprising a polypeptide described herein and a topically-acceptable carrier. The present invention also provides a topical composition comprising a polypeptide described herein and a topically-acceptable carrier.

The composition of the present invention may comprise one or more further active agent, preferably for promoting wound healing and/or treating or preventing a skin condition. Moreover, the present invention provides a composition for cosmetic use comprising a polypeptide as described herein and a cosmetically acceptable carrier, excipient or diluent. Further, the present invention provides a cosmetic composition comprising a polypeptide as described herein and a cosmetically acceptable carrier, excipient or diluent. Additionally, the present invention provides a pharmaceutical composition comprising a polypeptide as described herein and a pharmaceutically acceptable carrier.

In some embodiments, the composition further comprises one or more lipids and/or one or more further active agents. The polypeptide as described herein has been identified as having antioxidant activities and therefore in some embodiments, the isolated or purified polypeptide protects the one or more lipids and/or the one or more further active agents from oxidative damage.

Further provided by the present invention is the use of a polypeptide as described herein for the manufacture of a topical composition, cosmetic composition, and/or pharmaceutical composition.

The present invention provides a method for promoting wound healing, or treating or preventing a skin condition in a subject (such as a mammal), comprising administering to the mammal, to the wound, or to a site of the skin condition an effective amount of a polypeptide comprising a polypeptide as described herein or a composition as described herein. Preferably the polypeptide or composition is administered to the skin of the subject. In some embodiments of the method the invention may be one or more of: skin aging, oxidative damage; damage induced by sunlight exposure; ultraviolet radiation induced damage; UVB induced damage; UVA induced damage; and/or DNA damage.

In some embodiments of the method, the one or more of the above skin conditions are characterised by CPD and/or 8-OHdG formation, particularly DNA damage characterised by CPD and/or 8-OHdG formation or ultraviolet induced damage characterised by CPD and/or 8-OHdG formation.

The present invention also provides a method for inhibiting matrix metalloproteinase 1 (MMP1) activity in a cell, or in the skin of a subject, comprising treating the cell or the skin with a polypeptide or a composition as described herein.

Further provided is a method for inhibiting CREB phosphorylation in a cell, the method comprising treating the cell with a polypeptide or composition as described herein. Additionally provided is a method for inhibiting 8-OHdG formation in a cell, comprising treating the cell with a polypeptide or composition as described herein. A method is further provided for inhibiting CPD formation in a cell, the method comprising treating the cell with a polypeptide or composition as described herein.

### BRIEF DESCRIPTION OF DRAWINGS

Certain embodiments are illustrated by the following Figures. It is to be understood that the following description is for the purpose of describing particular embodiments only and is not intended to be limiting with respect to the description.

FIG. 1 shows the effect of the polypeptide IK34720 (SEQ ID NO: 3—RSKAKNPLYRRRRRRRRR) on wound healing.

FIG. 2 shows the effect of the polypeptide IK236770 (SEQ ID NO: 4—rrrrrrrrrylpnkaksr) on wound healing.

FIG. 3A shows the effect of the polypeptide IK34720 (SEQ ID NO: 3) on preventing UV-irradiation induced phosphorylation of Cyclic-AMP-responsive element-binding protein (CREB), and FIG. 3B shows the effect of the polypeptide IK34720 (SEQ ID NO: 3) on restoring UV-induced suppression of TGFβ receptor II (TGFβRII)).

FIG. 4 shows the effect of the polypeptide IK34720 (SEQ ID NO: 3) on ATP levels in primary keratinocytes exposed to solar simulated UV-irradiation.

FIG. 5 is immunohistology illustrating the effect of the polypeptide IK34720 (SEQ ID NO: 3) on inhibiting UV-induced oxidative stress in the skin.

FIG. 6 shows the effect of the polypeptide IK34720 (SEQ ID NO: 3) on inhibiting UV-induced oxidative stress in the skin.

FIG. 7 is immunohistology illustrating the effect of the polypeptide IK34720 (SEQ ID NO: 3) on UV-induced DNA damage, as demonstrated by formation of Cyclobutane pyrimidine dimers (CPDs) in vitro.

FIG. 8 shows the effect of the polypeptide IK34720 (SEQ ID NO: 3) on UV-induced DNA damage, as demonstrated by formation of CPDs in vitro.

FIG. 9 is immunohistology illustrating the effect of the polypeptide IK34720 (SEQ ID NO: 3) on UV-induced DNA damage, as demonstrated by formation of CPDs in vivo.

FIG. 10 shows the effect of the polypeptide IK34720 (SEQ ID NO: 3) on UV-induced DNA damage, as demonstrated by formation of CPDs in vivo in a murine model.

FIG. 11 illustrates the effects of the polypeptide IK34720 (SEQ ID NO: 3) on cell apoptosis following UV-irradiation.

FIG. 12 is immunohistology illustrating the effect of the polypeptide IK34720 (SEQ ID NO: 3) on UV-induced DNA damage, as demonstrated by formation of CPDs in human skin explants.

FIG. 13 shows the effect of the polypeptide IK34720 (SEQ ID NO: 3) on UV-induced DNA damage, as demonstrated by formation of CPDs in human explants.

FIG. 14 illustrates the effect of the polypeptide IK34720 (SEQ ID NO: 3) on the number of apoptotic cells in human skin explants 3 hrs after UV-irradiation.

FIG. 15 illustrates the effect of the polypeptide IK34720 (SEQ ID NO: 3) on the number of apoptotic cells in human skin explants 24 hrs after UV-irradiation.

FIG. 16 shows the effect of the polypeptide IK34720 (SEQ ID NO: 3) on MMP-1 activity.

FIG. 17 shows the effect of the polypeptide IK34720 (SEQ ID NO: 3) on the percentage of MMP-1 positive cells in human skin explants 3 hours after UV-irradiation.

FIG. 18 shows the effect of the polypeptide IK34720 (SEQ ID NO: 3) on the percentage of MMP-1 positive cells in human skin explants 3 hours after UV-irradiation.

FIG. 19 shows the effect of the polypeptide IK34720 (SEQ ID NO: 3) on Thiol Redox State (TRS) in primary fibroblast lysates.

FIG. 20 shows the effect of the polypeptide IK34720 (SEQ ID NO: 3) on the melanin content of cells.

### DETAILED DESCRIPTION

The nucleotide and polypeptide sequences referred to herein are represented by a sequence identifier number (SEQ ID NO:). A summary of the sequence identifiers is provided in Table 1. A sequence listing is also provided as part of the specification.

TABLE 1
Table of Sequence Listings

| Sequence | | |
|---|---|---|
| ID Number | | |
| (SEQ ID) | length | Sequence |
| SEQ ID NO: 1 | 10 | RSKAKNPLYR |
| SEQ ID NO: 2 | 10 | rylpnkaksr |
| SEQ ID NO: 3 | 18 | RSKAKNPLYRRRRRRRRR |
| SEQ ID NO: 4 | 18 | rrrrrrrrrylpnkaksr |

As will be described in more detail below, the present inventors have demonstrated that a polypeptide comprising the amino acid sequence RSKAKNPLYR (SEQ ID NO: 1), or the dextro-reverso form of the amino acid sequence (i.e. rylpnkaksr—SEQ ID NO:2), and a polyarginine amino acid sequence region, can treat a range of skin conditions such as promoting wound healing, treating or preventing aging, and can also treat and/or prevent the effects of UV-induced skin changes and cellular damage.

As used herein, the terms “IK34720”, “IK” or “IK-3” are used interchangeably to refer to SEQ ID NO: 3 (RSKAKNPLYRRRRRRRRR).

As used herein, the term “IK236770” is used interchangeably to refer to SEQ ID NO: 4 (rrrrrrrrrylpnkaksr).

The term “skin” as used herein, refers to at least the epidermis and dermis. As such, when used in relation to a skin condition, skin damage or skin wound, the term should be construed as not excluding conditions, damage or wounds that encompass additional tissues (such as the subcutaneous tissue) in addition to the epidermis and dermis.

The term “polypeptides” as used in the present specification refers to molecules composed of amino acid monomers, typically linked by amide bonds. The term includes a ‘pro-drug’ of the polypeptides, charged and non-charged forms of the polypeptides, a pharmaceutically acceptable salt of the polypeptides, and any other variant, derivative or modification to the polypeptides, including modifications to the backbone and/or termini of the polypeptides, which retain functional activity in the methods and uses of the present disclosure.

Further, the term polypeptide, as used herein, should not be interpreted as implicitly specifying a maximum length of the number of amino acids that can form the molecule. However, in some embodiments, when specified, the maximum length of the polypeptides is 50 amino acids. In some embodiments, the maximum length is 45 amino acids. In some embodiments, the maximum length of the polypeptides is 40 amino acids. In some embodiments, the maximum length is 35 amino acids. In some embodiments, the maximum length is 30 amino acids. In some embodiments, the maximum length is 25 amino acids. In some embodiments, the maximum length is 20 amino acids. In some embodiments, the maximum length is 18 amino acids.

In some embodiments, the polypeptide is an isolated or purified polypeptide.

Methods of “isolation” and “purification” of a polypeptide produced by natural or recombinant techniques are known in the art, for example in C-H Lee, A Simple Outline of Methods for Protein Isolation and Purification, Endocrinology and Metabolism; 2017, March; 32(1): 18. Further, the terms “isolated” or “purified” include synthesised and other artificially produced polypeptides. Methods for synthesising polypeptides are known in the art. Generally, polypeptides are chemically synthesized by the condensation reaction of the carboxyl group of one amino acid to the amino group of another. Chemical synthesis of polypeptides can be carried out using solution-phase techniques or solid-phase techniques. Synthetic techniques can allow for the production of polypeptides incorporating unnatural amino acid polypeptides, backbone modification and synthesis of D-isomers.

As used herein, the term polyarginine refers to a sequence of contiguous arginine amino acids. In some embodiments the polyarginine amino acid sequence region consists of 2 to 20 arginine residues. In some embodiments, the polyarginine amino acid sequence region consists of 4 to 20 arginine residues. In some embodiments, the polyarginine amino acid sequence region consists of 5 to 15 arginine residues. In some embodiments, the polyarginine amino acid sequence region consists of 5 to 12 arginine residues. In some embodiments, the polyarginine amino acid sequence region consists of 5 to 10 arginine residues. In some embodiments, the polyarginine amino acid sequence region consists of 5 to 9 arginine residues. In some embodiments, the polyarginine amino acid sequence region consists of 6 to 8 arginine residues. In some embodiments, the polyarginine amino acid sequence region consists of 7 or 8 arginine residues. In some embodiments, the polyarginine amino acid sequence region consists of 8 arginine residues.

In one aspect of the isolated or purified polypeptides described herein, the amino acid sequence RSKAKNPLYR (SEQ ID NO: 1), or the dextro-reverso form of the amino acid sequence rylpnkaksr (SEQ ID NO: 2), is adjacent the polyarginine amino acid sequence region.

In another aspect of the isolated or purified polypeptides described herein, the isolated or purified polypeptide further comprises a linker region in between the amino acid sequence RSKAKNPLYR (SEQ ID NO: 1), or the dextro-reverso form of the amino acid sequence (rylpnkaksr; SEQ ID NO: 2), and the polyarginine amino acid sequence region. Linkers, such as polypeptide linkers, are known in the field.

In some embodiments, the polyarginine amino acid sequence region is N-terminal and/or C-terminal of the amino acid sequence RSKAKNPLYR (SEQ ID NO: 1), or the dextro-reverso form of the amino acid sequence (i.e. rylpnkaksr—SEQ ID NO:2). For example, in some embodiments, the polyarginine amino acid sequence region is C-terminal of RSKAKNPLYR (SEQ ID NO: 1). In some embodiments, the polyarginine amino acid sequence region is N-terminal of rylpnkaksr (SEQ ID NO:2).

In some embodiments, the polypeptide comprises the amino acid sequence RSKAKNPLYRRRRRRRRR (SEQ ID NO: 3). In some embodiments, the polypeptide consists of the amino acid sequence RSKAKNPLYRRRRRRRRR (SEQ ID NO: 3).

In some embodiments, the polypeptide comprises the amino acid sequence rrrrrrrrrylpnkaksr (SEQ ID NO: 4). In some embodiments, the polypeptide consists of the amino acid sequence rrrrrrrrrylpnkaksr (SEQ ID NO: 4).

In some embodiments, the polypeptides of the invention are modified. In some embodiments, the modification may be a modification that alters the pharmacological properties of the polypeptides. In some embodiments, the modification increases the half-life of the composition or polypeptides of the invention. In some embodiments, the modification may increase the bioactivity of the polypeptides (and/or the composition of the invention). In some embodiments, the modification may be a modification that increases selectivity of the polypeptides or compositions of the invention.

In one embodiment, the modification is the addition of a protecting group. The protecting group may be an N-terminal protecting group, a C-terminal protecting group or a side-chain protecting group. The polypeptides of the present invention may have one or more of these protecting groups. The person skilled in the art is aware of suitable techniques to react amino acids with these protecting groups. These groups can be added by preparation methods known in the art. The groups may remain on the polypeptides or may be removed prior to use or administration. The protecting group may be added during synthesis.

In some embodiments, the polypeptide is amidated at its C-terminus. Amidation refers to the process of N-oxidative cleavage of glycine-extended substrates by sequential endo- and exoproteolysis. Methods are known in the art for producing amidated polypeptides in vitro, such as: enzymatic amidation; chemical modification of the C-terminus of recombinantly produced polypeptides and proteins; use of amide resins in solid-phase polypeptides synthesis; use of carboxypeptidase in the presence of ammonia; and conversion of the C-terminus of polypeptides to the methyl ester and addition of ammonia at low temperature. Examples of the disclosure of suitable techniques include DJ Merkler, C-terminal amidated polypeptides: production by the in vitro enzymatic amidation of glycine-extended polypeptides and the importance of the amide to bioactivity; Enzyme Microbial technology, 1994, June; 16(6): 450-6 and V Čeřovský and M-R Kula C-Terminal polypeptides Amidation Catalyzed by Orange Flavedo polypeptides Amidase; Angewandte Chemie, 1998, August; 37(13-14): 1885.

Amidation of the C-terminus results in the C-terminal end being uncharged, so the modified polypeptides more closely mimic a native protein. This can have a series of advantages including an enhanced ability of the polypeptide to enter a cell; an improvement in the metabolic stability of the polypeptide in vivo; a decrease in the in vivo enzymatic degradation of the polypeptides by aminopetidases, exopeptidases, and synthetases; and an improvement of the shelf-life of the polypeptides.

As is known in the art, alpha amino acids include a chiral carbon at the alpha position. Consequently, all alpha amino acids, with the exclusion of glycine, can exist in either of two enantiomers, being the L- or D-isomers. Generally, only L-amino acids are manufactured in mammalian cells and incorporated into proteins. D-amino acids can be artificially synthesised or may be found in bacterial proteins. The L and D convention is not used to directly refer to the stereochemistry of the amino acids, but rather to the optical activity of the isomer of glyceraldehyde from which that amino acid can be synthesized (D-glyceraldehyde is dextrorotatory; L-glyceraldehyde is levorotatory).

In some embodiments, the polypeptide of the present invention comprises L-amino acids. In some embodiments the polypeptide of the present invention comprises only L-amino acids. In some embodiments the polypeptide of the present invention comprises D-amino acids. In some embodiments the polypeptide of the present invention comprises only D-amino acids. In some embodiments the polypeptide of the present invention comprises L-amino acids and D-amino acids.

### Compositions

To facilitate application of the polypeptides, the polypeptides can be formulated into a composition. As defined herein a composition includes any admixture of components together with the polypeptides. In some forms, the composition is composed to stabilise the polypeptides and/or protect the polypeptides and/or improve the application of the polypeptides. In some embodiments, the polypeptides can help stabilise another component of the composition, and/or protect another component of the composition, and/or improve the application of another component of composition.

Many compositions contain biological components, such as lipids, enzymes, proteins, polypeptides, elastin, collagen and fibrin. Further, compositions generally include an aqueous solvent. Biological components, in the presence of water, will typically be oxidised over time as a result of the hydrolytic activity of the water. This leads to degradation of the biological components and diminishes their function within the composition. Therefore, it may be advantageous to include additional components in the composition which can reduce oxidation of these biological components, such as a polypeptide of the present invention.

The problem is particularly exacerbated in an oil-in-water carrier, most commonly used in skin-care compositions. There is significant diffusion of oxygen at the water-oil interface in these compositions, which can result in serious degradation of biologically-active components, especially sensitive compounds encapsulated in the oil phase.

Accordingly, there is provided a composition containing the polypeptides of the present invention. In some embodiments, the composition contains a polypeptide of the present invention and at least one biological, or oxidisable, component. In some embodiments, the biological or oxidisable component is selected from the group consisting of: a lipid, an enzyme, a protein, a polypeptide, elastin, collagen and fibrin. In some embodiments, the composition comprises one or more lipids or one or more further active compounds. In some embodiments of the composition, the polypeptide in accordance with the present invention protects one or more lipids and/or one or more further active agents, or components, from oxidative damage.

A composition can be any suitable composition, and will be adapted for its particular use by a person skilled in the art. For example, the composition may be formulated for use in in vitro experiments or in vivo administration. In some preferred embodiments, the composition is a topical composition. In some embodiments, the composition is a pharmaceutical composition, preferably a topical pharmaceutical composition. In some embodiments the composition is a cosmetic composition, preferably a topical cosmetic composition.

### Topical Composition

Importantly, the present inventors have demonstrated that the polypeptides are active when applied topically. For example, the present inventors have demonstrated that polypeptides described herein are able to promote wound healing, and decrease skin damage, when applied topically to skin in vivo or ex vivo. Accordingly, in one aspect, the present invention provides a composition for topical use, or a topical composition, comprising a polypeptide in accordance with the present invention and a topically acceptable carrier, diluent or excipient. In another aspect the present invention provides the use of a polypeptide in accordance with the present invention for the manufacture of a composition for topical use, or a topical composition. Preferably the composition for topical use is used for treating skin conditions, such as those described herein.

The term “topical composition” or “composition for topical use” refers to a composition that is formulated for topical administration, being the application primarily to keratinous tissue, primarily the skin, but may include hair and nails. Topical generally relates to delivery to the skin, but can also mean delivery to lumen spaces lined with epithelial cells, for example mucosal tissue such as the lips, mouth etc.

Formulations for topical delivery are described in D Osborne and A Aman (eds),1990, Topical drug delivery formulations, CRC press Taylor & Francis and D Bhowmik et al. (2012) Recent Advances In Novel Topical Drug Delivery System, the Pharma innovation, 1:9, 12-31. Acceptable carriers or diluents for therapeutic use are well known in the pharmaceutical art, and are described, for example, in Tarun Garg, Goutam Rath & Amit K. Goyal (2015) Comprehensive review on additives of topical dosage forms for drug delivery, Drug Delivery, 22:8, 969-987.

A suitable form for topical administration comprises liquid, ointment, cream, gel, hydrogel, pomade, liniment, lotion, emulsion, spray, aerosol, drops or powder. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Drops or liquid sprays may be formulated with the polypeptides of the invention in an aqueous, or non-aqueous, base also comprising one or more of: dispersing agents, solubilizing agents or suspending agents. Drops can be delivered via a simple eye dropper-capped bottle, via a plastic bottle adapted to deliver liquid contents drop-wise, or via a specially shaped closure. Liquid sprays can be pumped or are conveniently delivered from pressurized aerosols and can be delivered via a targeted spray opening such as a manipulable tube or can be delivered via a spray aperture which spreads the liquid over a uniform area. Further, a solubilised form of the polypeptide can be contained in an absorbent medium which can then be placed or rubbed on the skin (for example a fabric wipe containing solubilised polypeptide).

In some forms of the invention, the polypeptides of the invention can be delivered via patches, plasters, poultices or bandages for dermal administration. Alternatively, the polypeptide can be formulated to be part of an adhesive polymer, such as polyacrylate or acrylate/vinyl acetate copolymer.

The topical composition may optionally comprise ingredients such as water, oils, salts, fragrances, perfumes, colorants, stabilisers, emulsifiers, propellants, additives, preservatives or preserving agents, anti-oxidants, surfactants, thickeners and other excipients normally used in topical compositions.

In some embodiments, the composition for topical use or the topical composition, comprises one or more further active agents for promoting wound healing, or treating or preventing a skin condition.

The term “treatment” and related terms as used herein refer to obtaining a desired pharmacologic and/or physiologic effect that is therapeutic in nature. For example, the effect may be therapeutic in terms of improving the condition of the subject, ameliorating, relieving and/or slowing the progression of one or more symptoms in the subject, a partial or complete stabilization of the subject, or a cure in the subject.

The term “preventing” and related terms as used herein refer to obtaining a desired pharmacologic and/or physiologic effect that is prophylactic in nature. For example, the effect may be a complete or partial prevention of a disease, condition, state or symptom in the subject, or the complete or partial prevention of the progression or occurrence of symptoms or pathology in the subject.

Topical compositions in accordance with the present invention can be divided into cosmetic composition (or compositions intended solely for cosmetic uses), and pharmaceutical compositions (or compositions which are intended to be used, inter alia, for the prevention or treatment of a disease, ailment or condition).

### Cosmetic Composition

The term “cosmetic composition” when used herein relates to a composition that can be used for cosmetic purposes, personal care and/or hygiene purposes. It will be appreciated that the composition may have more than one cosmetic purpose and may be used for more than one of these purposes at the same time. The term “cosmetic composition” when used herein can include but is not limited to, moisturising creams, facial and body powder and the like. Further, cosmetic compositions may include nail polish, compacts, solids, pencils, lipstick, mascara, rouge, foundation, blush, eyeliner or the like.

The US Food and Drug Administration (FDA) considers a cosmetic composition is defined by The Federal Food, Drug, and Cosmetic Act (FD&C Act), which defines cosmetics by their intended use, as “articles intended to be rubbed, poured, sprinkled, or sprayed on, introduced into, or otherwise applied to the human body . . . for cleansing, beautifying, promoting attractiveness, or altering the appearance” [FD&C Act, sec. 201(i)]. Among the products included in this definition are skin moisturizers, perfumes, lipsticks, fingernail polishes, eye and facial makeup preparations, cleansing shampoos, permanent waves, hair colours, and deodorants, as well as any substance intended for use as a component of a cosmetic product.

Some products may be considered as both cosmetics and pharmaceutical, depending on their intended use. In such situations, the term “cosmetic” should be taken to exclude the pharmaceutical or therapeutic application of the composition.

Acceptable carriers or diluents for cosmetic uses are known in the art, and are described, for example, in AO Barel, M Payne and HI Mailbach (Eds) (2014) Handbook of Cosmetic Science and Technology, 4^{th} Edition, CRC Press Taylor & Francis Group, USA.

A reference to a “non-therapeutic”, or “cosmetic”, methods and compositions throughout this specification should be taken to exclude any potential therapeutic methods or applications which may otherwise be encompassed by the claim(s). In some embodiments, a reference to a “non-therapeutic” or “cosmetic” method composition may allow for the specific disclaiming of a method of treatment by therapy performed on a human, or a composition for therapeutic treatment of a human. It is to be understood that while the compositions and methods of the present application may be biologically active, such as reducing photoaging or reducing the secretion of proteases or other enzymes, these are not to be considered a method of therapy, or compositions for therapy, as there is no therapeutic indication or associated disease state.

### Pharmaceutical Composition

In one aspect, the present invention provides a composition for pharmaceutical use, or a pharmaceutical composition, comprising a polypeptide in accordance with the present invention and a pharmaceutically acceptable carrier or diluent. In another aspect the present invention provides the use of a polypeptide in accordance with the present invention for the manufacture of a medicament for topical use, or a pharmaceutical composition.

The US Food and Drug Administration (FDA) considers pharmaceuticals as defined by The Federal Food, Drug, and Cosmetic Act (FD&C Act), in part, by their intended use, as “articles intended for use in the diagnosis, cure, mitigation, treatment, or prevention of disease” and “articles (other than food) intended to affect the structure or any function of the body of man or other animals” [FD&C Act, sec. 201(g)(1)].

The composition also includes a pharmaceutically acceptable carrier, excipient or diluent. The phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

Suitable carriers include, but are not limited to, substantially inert solid, semi-solid or liquid fillers, diluents, excipients, encapsulating materials or formulation auxiliary of any type. An example of a pharmaceutically acceptable carrier is physiological saline or phosphate-buffered saline (PBS). Other physiologically acceptable carriers and their formulations are known in the art. Some examples of materials which can also serve as pharmaceutically acceptable carriers or excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN 80; buffering agents such as magnesium hydroxide and aluminium hydroxide; alginic acid; pyrogen-free water; isotonic saline; Ringer's solution; ethyl alcohol; and phosphate buffer solutions, as well as other non-toxic compatible lubricants such as sodium lauryl sulphate and magnesium stearate, as well as colouring agents, releasing agents, coating agents, sweetening, flavouring and perfuming agents, preservatives and antioxidants, suspending agent(s) and solubilising agent(s) can also be present. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. In one embodiment the present invention provides preservatives and/or stabilizers. Examples of preservatives include sodium benzoate, sorbic acid and esters of p-hydroxybenzoic acid. Antioxidants and suspending agents may be also used.

Acceptable carriers are further described in the art, for example, in A. R. Gennaro (ed) 2015, Remington's Pharmaceutical Sciences, 14^{th} Edition, Mack Publishing Co. The choice of pharmaceutical carrier, excipient or diluent can be selected with regard to the intended route of administration and standard pharmaceutical practice.

“Pharmaceutical compositions” refers to an admixture of the polypeptides of the present invention in combination with one or more pharmaceutically acceptable diluents, excipients or carriers. In some forms the components of the admixture form an interworking relationship wherein the functionality of the active ingredient(s) (such as the polypeptide of the invention) is enhanced. For example, the admixture may increase the bioavailability of the polypeptide by improving or prolonging contact with the site of action such as the epidermis, dermis or wound; by reducing evaporation of the pharmaceutical composition; by improving stability of the polypeptide; by preventing enzymatic degradation or oxidation of the polypeptide; and/or by interacting with the polypeptide to create a synergistic relationship whereby the interaction of elements when combined produce a total effect that is greater than the sum of the individual elements. Typically, this synergistic relationship is manifested in a greater biological effect than can be achieved by the sum of the two components administered alone.

The pharmaceutical compositions may be for human or animal usage in human and veterinary medicine. Examples of such suitable excipients for the various different forms of pharmaceutical compositions described herein may be found in PJ Sheskey, WG Cook and CG Cable (eds) 2017, Handbook of Pharmaceutical Excipients, 8th Edition, APhA/Pharmaceutical Press, USA.

The term “effective amount” (for example a “therapeutically effective amount” or a “cosmetically effective amount”) as used herein refers to an amount that allows the achievement of the contemplated end, i.e. the healing of a wound, decrease in UV related damage and a decrease in photoaging. Said “effective amount” will vary from subject to subject, depending on the age and general condition of the individual and with the factors such as the particular condition being treated, the duration of the treatment, previous treatments and the nature and pre-existing duration of the condition (e.g. acute vs chronic wound).

Specifically, an effective amount of an agent defines an amount that can be administered to a subject without excessive or non-tolerable toxicity, irritation, allergic response, or other problem or complication, commensurate with a reasonable benefit/risk ratio, but one that is sufficient to provide the desired effect as assessed by an appropriate technique such as those disclosed throughout this specification. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms. A therapeutic result need not be a complete amelioration of the condition (i.e. a cure).

The polypeptides of the invention may be administered, or formulated in a composition, in the form of a pharmaceutically acceptable salt. The pharmaceutically acceptable salts of the present invention can be derived from the parent polypeptide which contains a basic, acidic or metallic moiety by conventional chemical methods. Generally, such salts can be prepared by reacting acidic or basic forms of the polypeptide with a stoichiometric amount of an appropriate counter base or acid in an aqueous or organic solvent. Generally, nonaqueous solvents such as ether, ethyl acetate, ethanol, isopropanol, or acetonitrile are preferred. Examples of acid addition salts include organic acids such as acetic, lactic, palmoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. Other pharmaceutically acceptable salts are contemplated. Lists of suitable salts are found in Remington's Pharmaceutical Sciences, 17th ed., Mack Publishing Company, Easton, Pa., US, 1985, p. 1418, and Stahl et al, Eds, “Handbook of Pharmaceutical Salts Properties Selection and Use”, Verlag Helvetica Chimica Acta and Wiley-VCH, 2002.

### Skin Conditions

As discussed above, the present inventors have demonstrated that the polypeptides and compositions of the present invention are useful in treating skin conditions. Particularly, the polypeptides and compositions are useful in preventing or treating skin conditions associated with damage to the skin, generally as a result of an insult to the skin. Particularly exemplified skin conditions include wound healing, treating or preventing UV damage to the skin, treating or preventing skin aging including photoaging, treating or preventing oxidative stress and reactive oxygen species in skin cells, treating or preventing damage to the extracellular matrix (ECM) of the skin and treating or preventing DNA damage to cells of the skin.

For example, Example 1 demonstrates that the polypeptides IK34720 (SEQ ID NO: 3—RSKAKNPLYRRRRRRRRR) and the dextro-reverso sequence IK236770 (SEQ ID NO: 4—rrrrrrrrrylpnkaksr) promotes wound healing when applied topically, compared to untreated controls. Example 2 demonstrates that the polypeptide IK34720 (SEQ ID NO: 3) inhibits UV-induced phosphorylation of serine at position 133 of CREB (CREB S-133) in primary keratinocytes when applied following keratinocyte exposure to solar simulated UV-irradiation. Example 2 also shows that UV treatment supresses TGFβRII levels, and the polypeptide IK34720 (SEQ ID NO: 3) restored TGFβRII expression in UV treated cells. Example 3 demonstrates the polypeptide IK34720 (“IK”, SEQ ID NO: 3) enhanced ATP levels in primary keratinocytes following exposure to solar simulated UV, to a similar level as Calcitriol. Example 4 demonstrates the polypeptide RSKAKNPLYRRRRRRRRR, when applied topically to skin following UV-irradiation, decreases oxidative stress in skin cells induced by UV-irradiation. Example 5 demonstrates the polypeptide RSKAKNPLYRRRRRRRRR, when applied topically to skin following UV-irradiation, decreases DNA damage, as measured by the formation of CPD nuclei in skin cells induced by UV-irradiation. Example 6 demonstrates topical application of the polypeptide IK34720 (SEQ ID NO: 3) reduced the number of apoptotic keratinocytes following UV-irradiation. Example 7 demonstrates the polypeptide RSKAKNPLYRRRRRRRRR, when applied topically to skin following UV-irradiation, can reduce the formation of CPD in human skin explants following acute UV-irradiation at 3 hours post irradiation. Example 8 demonstrates the polypeptide IK34720 (SEQ ID NO: 3) inhibits MMP-1 activation in a dose dependent manner, and when applied topically to skin following UV-irradiation, decreases MMP1 expression induced by UV-irradiation, indicating the polypeptide IK34720 (SEQ ID NO: 3) can be used to reduce photoaging over time. Example 9 demonstrates the polypeptide RSKAKNPLYRRRRRRRRR, decreases UV-induced oxidative damage of cell-derived lipids in dermal skin fibroblast cell lysates. Example 10 demonstrates the polypeptide RSKAKNPLYRRRRRRRRR, decreases melanin induction by UVB.

Accordingly, the present invention provides a method for treating a skin condition comprising administering to the skin, or cells of the skin, an effective amount of a polypeptide or composition as described herein.

As used herein, the terms “administering” or “providing” include administering the polypeptides, or administering a prodrug, or a derivative of the polypeptides, or a composition containing any one of the aforementioned polypeptides, that will result in the delivery of an effective amount of the active polypeptides within the body of the subject, or within the target cell.

### Wound Healing

As discussed above, the present inventors have demonstrated that the polypeptides of the present invention are useful in promoting the healing of wounds. Accordingly, in an aspect of the present invention, there is provided a polypeptide according to the present invention for promoting wound healing. In another aspect of the present invention there is provided a method for promoting wound healing in a mammal, comprising administering to the mammal an effective amount of a polypeptide or a composition in accordance with the present invention.

In this specification, the term “wound healing” is used to describe all the different steps involved in the healing of wounds and includes the augmentation and acceleration of the healing of the wound. It therefore includes the steps of forming a clot that plugs the defect, invasion of the clot by inflammatory cells and then of fibroblasts and capillaries to form a contractile granulation tissue that draws the wound margins together, and migration forward of the cut epidermal edges to cover the denuded wound surface.

The term “wound” as used herein refers to any wound to the tissue of the body. In a preferred form the term “wound” relates to dermal wounds where the skin is disrupted forming a tear, cut, puncture, incision, laceration, abrasion, rip, slash, scratch, slit, burn, rupture or ulceration in the skin of an animal.

Such wounds can be categorised as acute or chronic. Chronic wounds are defined as wounds that have failed to repair in a timely and orderly process of repair for at least a period of 3 months. Chronic wounds can be identified by the presence of a raised, hyperproliferative but non-advancing wound margin. Fibroblasts derived from the wound bed of chronic wounds of various aetiologies represent a senescent, premature, or differentiated phenotype, which do not respond or respond inefficiently to normal stimulatory messages.

All wound types have the potential to become chronic. Chronic wounds are traditionally divided etiologically, which may inform the treatment of the chronic wound. Further, treatment of the chronic wounds may also be accompanied by treatment of the underlying condition which may have instigated, or contributed to the formation of, the wound such as venous insufficiency, arterial perfusion, diabetes, unrelieved pressure as well as systemic factors such as nutritional status (Type I or Type II diabetes), immunosuppression and infection.

Common chronic wounds are venous ulcers, which usually occur in the legs and mostly affect the elderly, diabetic ulcers which are another major cause of chronic wounds, pressure ulcers, which usually occur in people with conditions such as paralysis that inhibit movement of body parts that are commonly subjected to pressure such as the heels, shoulder blades and sacrum, corneal ulcers, most commonly caused by an infection with bacteria, viruses, fungi or amoebae. Other types of chronic wounds may be due to causes such as ischemia and radiation poisoning.

Acute wounds can be categorised as any wound that does not meet the classification of a chronic wound. As such, acute wounds can be defined as wounds less than 3 months since the instigation of the wound site, often showing signs of wound healing within the first 4 weeks since instigation.

Acute wounds may be classified into different types, according to the object that caused the wound. For example, incisions or incised wounds, lacerations, abrasions and grazes, burns, puncture or penetration wounds.

In various embodiments the polypeptides or compositions of the invention may be used with, or the methods of the invention may further comprise, an adjunct treatment. The adjunct treatment may include a debridement treatment such as the use of papain or other debridement agents known in the art, applications of a moist wound bed, or management of microorganism loads via anti-biotics or antifungals.

There are various techniques known in the art for measuring wound healing. These include but are not limited to the following.

Ruler technique—The surface area of a wound can be approximated by multiplying the greatest length and perpendicular width measurements. This technique is limited by the non-uniform nature of the shape of most wounds and as such this technique does not accurately predict the area of wounds having irregular shapes, which are large or deep. An alteration of this technique is to also measure the depth of the wound to predict the total wound volume. However, the accuracy of this technique is limited for the same reasons as discussed above.

Acetate Tracing and Planimetry—An alternative method for estimating wound area is acetate tracings and contact planimetry. This technique involves placing a transparent sheet across the wound surface and then tracing its margins. The area of the wound is then determined manually by placing the tracing over a grid and counting the number of squares within the circumscribed area, or by using computer image analysis to accurately quantify area.

Wound area estimation can also be estimated in non-contact manner such as non-contact planimetry. In this technique a target plate or scale gauge is placed in the same plane as the wound and an image is captured. Digital image analysis is undertaken to estimate the area of the wound with reference to the target plate/scale.

Three-dimensional estimation of wounds—More complicated three-dimensional topography measurement of wounds can be performed using structured light or laser light. These techniques, which are known in the art, use digital cameras and projected laser beams that distort with the curvature and depth of the wound surface.

### Skin Damage and Skin Conditions

Importantly, the present inventors have further demonstrated that the polypeptides of the present invention can be used to prevent or treat damage caused to the skin by an insult to the skin (such as non-ionizing radiation damage, e.g. UV damage) or by a medical condition of the skin. Accordingly, in an aspect, there is provided polypeptides according to the present invention for treating or preventing a skin condition. In another aspect, there is provided compositions for treating or preventing a skin condition. In another aspect, there is provided a method for treating or preventing a skin condition in a mammal, comprising administering to the mammal an effective amount of a polypeptide, or a composition as described herein.

Moreover, in another aspect there is provided a method for treating or preventing damage in a cell, the method comprising treating the cell with polypeptides as described herein.

In some embodiments, the skin condition is skin damage. In some embodiments, the damage is induced by sunlight exposure. In some embodiments, the skin condition is ultraviolet (UV) radiation induced damage. In some embodiments, the UV-radiation is UVB radiation, and the damage is UVB induced damage. In some embodiments, the UV-radiation is UVA radiation, and the damage is UVA induced damage. In some embodiments, the skin condition is DNA damage. In some embodiments, the DNA damage is induced by UV-radiation, such as UVB or UVA radiation. In some embodiments the DNA damage is caused by UVB radiation. In some embodiments the DNA damage is caused by UVA radiation

In some embodiments, the skin condition is oxidative damage. In some embodiments, the damage is increased cellular apoptosis. In some embodiments, the damage is damage to the extracellular matrix (ECM) of the skin. In some embodiments, the ECM is a collagen, elastin, laminin or a Proteoglycans, such as dermatan sulphate and hyaluronan.

In some embodiments, the polypeptides, compositions and methods(s) disclosed herein inhibit DNA damage. In some embodiments, the polypeptides, compositions and methods(s) disclosed herein increases repair of ultraviolet radiation induced damage.

The term “treating” in the context of damage (such as damage caused by UV exposure) refers to reducing the relative level of any detrimental change caused in a cell, or tissue, as a result of the insult that caused the damage, when the polypeptides or compositions as described herein are applied, or the methods as described herein are performed, after the insult (e.g. UV-irradiation). Further, the term “prevention” in the context of damage refers to reducing the relative level of any detrimental change caused in a cell, or tissue, as a result of the insult that caused the damage, when the polypeptides or compositions as described herein are applied, or the methods as described herein are performed, before the insult, or before the induction of the detrimental change caused in a cell or a tissue as a result of the insult.

### UV Damage

The present inventors have demonstrated that polypeptides in accordance with the present invention, such as the sequence RSKAKNPLYRRRRRRRRR (SEQ ID NO: 3), when applied topically to skin following UV-irradiation, reduce DNA damage.

Ultraviolet radiation is typically defined as electromagnetic radiation having a wavelength of 10 nm to 400 nm. The most common source of environmental UV-radiation is from the sun where it makes up 10% of total light output, with the most of it filtered out by the atmosphere of the earth such that at ground level about 3% of electromagnetic radiation from the sun is UV-radiation.

UV emitted from the sun primarily can be divided into UVA, UVB and UVC radiation. UVA is categorised as having a wavelength of 315 nm to 400 nm; UVB is categorised as having a wavelength of 280 nm to 315 nm; and UVC is categorised as having a wavelength of 100 nm to 280 nm. At sea level about 95% of UV-radiation is UVA, and about 5% being UVB with the UVC radiation being filtered out by the atmosphere.

It has been demonstrated in animal models and human skin explants that UVB and UVA induce photo-damage to the skin, with UVA penetrating deeper than UVB. UV-induced DNA photoproducts are able to cause specific mutations (UV-signature) in susceptible genes, and as such are a causative agent in the induction of a range of skin cancers, including basal-cell carcinomas and melanomas.

As such, and as discussed above, the present invention provides polypeptides, compositions and methods for treating or preventing UV damage in the skin, in particular, in skin cells or in the ECM of the skin.

The types of damage caused by UV exposure are known in the art, and include (but are not limited to), DNA damage, apoptosis of cells, activation of intracellular signalling pathways by phosphorylation of cellular signalling molecules such as cAMP response element-binding protein (CREB) and generation of reactive oxygen species (ROS).

### DNA Damage and CPD Formation

There are a range of techniques known in the art for assessing DNA damage within a cell. These include (but are not limited to) the CometAssay®, PARP Universal Colorimetric Assay, Superoxide Dismutase Assay analysis of phosphorylated H2AX within cells, or production of cyclobutane pyrimidine dimers (CPDs).

During exposure of keratinocytes to UV-radiation, two forms of DNA damage are induced; cyclobutane pyrimidine dimers (CPDs), and pyrimidine photoproducts DNA damage. CPDs are highly mutagenic and are produced in substantial quantities by UV-radiation, particularly UVB radiation. These dimers can form between any two adjacent pyrimidines and can involve thymine, cytosine, or 5-methylcytosine. The mutagenicity of CPDs relates to their long persistence in skin cells. This allows time for deamination of the CPDs which, when the deaminated CPDs are bypassed by DNA polymerases, result in a mutagenic event.

The present inventors have demonstrated that polypeptides in accordance with the present invention, such as the sequence RSKAKNPLYRRRRRRRRR (SEQ ID NO: 3), when applied topically to skin following UV-irradiation, decreases CPD formation. Therefore, in some embodiments there is provided polypeptides, compositions(s) and methods for reducing damage characterised by CPD formation in a cell, particularly UV-induced damage. Furthermore, in some embodiments there are provided polypeptides, compositions(s) and methods for preventing damage characterised by CPD formation in a cell, particularly UV-induced damage.

In some embodiments, the damage is CPD formation in a cell. Accordingly, in some embodiments, the polypeptides, the compositions and the methods described herein reduce CPD formation in a cell, particularly following UV exposure. Further, in some embodiments, the polypeptides, the compositions and the methods described herein prevent increases in CPD formation in a cell, particularly following UV exposure. Accordingly, there is provided a method for inhibiting CPD formation in a cell (such as a skin cell), comprising treating the cell with a polypeptide or composition in accordance with the present invention.

### Oxidative Damage

Oxidative stress is the result of the imbalance between reactive oxygen species (ROS) formation and enzymatic and nonenzymatic antioxidants. A net imbalance can result in an accumulation of ROS within cells, and secreted from cells. The increase in ROS can cause a range of damage to cells and the extracellular matrix of a tissue (such as the skin) including DNA damage (for example double strand DNA breaks) and oxidative damage to both cellular and extracellular components, such as lipids and collagen.

The present inventors have demonstrated that polypeptides in accordance with the present invention, such as the sequence RSKAKNPLYRRRRRRRRR (SEQ ID NO: 3), when applied to UV irradiated skin, decreases oxidative stress within a cell. In particular, the polypeptides in accordance with the present invention, such as the sequence RSKAKNPLYRRRRRRRRR (SEQ ID NO: 3), when applied topically to skin following UV-irradiation inhibits 8-OHdG formation in the skin.

Accordingly, the invention provides polypeptides and compositions as described herein for treating or preventing oxidative stress in a cell, or oxidative damage to the skin. In a further aspect, the invention provides a method for treating or preventing oxidative damage to the skin of a subject, comprising administering (preferably topically) to the subject an effective amount of a polypeptide or composition in accordance with the present invention. In some embodiments, the oxidative damage is UV-radiation induced oxidative damage. In some embodiments of the method, the polypeptides or composition is administered before UV-irradiation. In some embodiments of the method, the polypeptides or composition is administered after UV-irradiation.

The invention also provides polypeptides and compositions as described herein for treating and/or preventing an increase in ROS in a cell. In a further aspect, the invention provides a method for treating or preventing increases in ROS concentrations in the skin of a subject, comprising administering (preferably topically) to the subject an effective amount of a polypeptide or composition in accordance with the present invention. In some embodiments, the increase in ROS follows UV-irradiation of the skin or cells. In some embodiments of the method, the polypeptides or composition is administered before UV-irradiation. In some embodiments of the method, the polypeptides or composition is administered after UV-irradiation.

Methods and techniques are known in the art to assess oxidative damage in a cell, for example I. Marrocco et al. Measurement and Clinical Significance of Biomarkers of Oxidative Stress in Humans. Oxidative Medicine and Cellular Longevity, 2017, Article ID 6501046. These techniques include, but are not limited to: fluorescent probes for detection of ROS and ROS inducing nitrogen species (e.g. DHR123, DCFH-DA, HE and C11-BODIPY); markers based on ROS-induced modifications, such as lipid oxidation (e.g. HNE, F2-IsoPs, MDA, alkenals and alkadienals), oxidation of DNA (e.g. 8-OHdG, 5-chlorocytosine, 5-chlorouracil, εdA and εdC) and oxidation of lipids (e.g. carbonils, 3-NO-Tyr, AOPP, ALEs, AGEs and oxLDL).

As disclosed above, the present inventors have demonstrated that polypeptides in accordance with the present invention can reduce the concentration of 8-OHdG in a cell following UV exposure, and UV-induced damage. Therefore, the present invention provides polypeptides or compositions as described herein for reducing 8-OHdG in a cell. In some embodiments, the 8-OHdG is induced by UV-irradiation of the cell (ultraviolet stress). Further provided are methods for reducing 8-OHdG in a cell, comprising treating the cell with a polypeptide or composition as described herein. In some embodiments of the method, the polypeptides or composition is administered before UV-irradiation. In some embodiments of the method, the polypeptides or composition is administered after UV-irradiation. In some embodiments, the method inhibits or reduces UVB induced damage, characterised by 8-OHdG formation. In some embodiments, the method inhibits or reduces UVA induced damage.

### DNA Damage and Cellular Apoptosis

The present inventors have demonstrated that polypeptides in accordance with the present invention, such as the sequence RSKAKNPLYRRRRRRRRR (SEQ ID NO: 3), when applied topically to skin, including human skin, following UV-irradiation reduced the number of apoptotic sunburn cells.

Damages caused to cells following an insult, such as UV-irradiation, can lead to cells undergoing programmed cell death via apoptosis. DNA damage caused by insults such as UV-irradiation and reactive oxygen species can regulate the function and activity of multiple apoptotic factors, such as p53, Ku70 and Ku86 (the Ku complex) and the MNR complex. This in turn can induce apoptosis in cells, with increased apoptosis being correlated with skin aging.

Accordingly, in one aspect the present invention provides polypeptides, compositions as described herein for reducing apoptosis in the skin (e.g. in one or more cells of the skin), or preventing apoptosis in a cell. In a further aspect, the invention provides a method reducing apoptosis in the skin, or preventing apoptosis in a cell, comprising administering (preferably topically) to the subject an effective amount of a polypeptide or composition in accordance with the present invention.

Methods are known in the art for assessing apoptosis in cells for example G Banfalvi, Methods to detect apoptotic cell death, Apoptosis, 2017 February; 22(2):306-323. These include, but are not limited to, analysis of: membrane alterations, DNA fragmentation, cytotoxicity and cell proliferation and mitochondrial damage. Further techniques include light-scattering flow cytometry, time-lapse microscopy perfusion assays and analysis of genotoxicity specific chromatin changes.

### Cellular Energy and DNA Repair

The present inventors have demonstrated that polypeptides in accordance with the present invention, such as the sequence RSKAKNPLYRRRRRRRRR (SEQ ID NO: 3), when applied topically to skin, including human skin, following UV-irradiation increases the levels of ATP in cells.

Vitamin D, which is produced in cells following UV-irradiation, is locally hydroxylated and protects cells against UV-induced damage. Further, the vitamin D steroid hormone 1alpha,25-dihydroxyvitamin D₃ (1,25(OH)₂D₃; Calcitriol) has been shown to increase the protective effects of vitamin D and reduce DNA damage, and improve DNA repair, in a variety of models.

Moreover, it has recently been shown that 1,25(OH)₂D₃ enhances glycolysis leading to energy-conserving processes. Critically, DNA repair is energy dependent and therefore an increase in intracellular energy, via increased Adenosine Triphosphate (ATP), can result in increased repair of CPDs and decreased oxidative damage. Therefore, the ability to increase the ATP concentration in a cell following UV-irradiation, should facilitate improved DNA repair.

Accordingly, in one aspect the present invention provides polypeptides and compositions as described herein for increasing the ATP levels in a cell. In some embodiments, the increase in ATP in a cell is following UV exposure. Further provided is a method of increasing the ATP level in a cell, comprising treating the cell with polypeptides or compositions as described herein. In some embodiments, the cell is treated before exposure to UV-radiation. In some embodiments the cell is treated after exposure to UV-radiation.

### CREB Activation

The present inventors have demonstrated that polypeptides in accordance with the present invention, such as the sequence RSKAKNPLYRRRRRRRRR (SEQ ID NO: 3) inhibits formation of phosphorylated cAMP response element-binding protein (pCREB) in primary keratinocytes, when applied following keratinocyte exposure to solar simulated UV-irradiation.

CREB is a cellular transcription factor. It binds to certain DNA sequences called cAMP response elements (CRE), thereby increasing or decreasing the transcription of the genes and has a role in a range of cellular pathways including cell proliferation, cell cycle progression, metabolism, DNA repair, cell differentiation and cell survival. CREB activation is indicated by phosphorylated at the serine at position 133. Phosphorylation at position 133 is increased as a result of UV-irradiation and is considered as a marker and contributing agent of UV-induced damage to cells. As such, inhibition of CREB phosphorylation may decrease UV-induced damage to keratinocytes, and hence decrease UV damage to skin.

Accordingly, in one aspect, the present invention provides polypeptides and compositions in accordance with the present invention for reducing CREB phosphorylation. Further provided is a method for inhibiting CREB phosphorylation in a cell, comprising treating the cell with polypeptides or compositions in accordance with the present invention.

### TGFbetaRII and Procollagen Synthesis

There are number of reports linking sunscreen protection with TGFB/Smad signalling and stimulation of procollagen synthesis (Rittie L & Fisher GJ, Ageing Res Reviews, 2002, 1(4): 705-720; Choi MS et al, J Dermatol Sci, 2007, 146(2): 127-137; Bora NS et al, Eur J Pharm Sci, 2018, 127: 261-275).

Moreover, it has also been reported that 8 hours post UV irradiation both TGFB2 and TGFBRII are reduced, and this parallels the reduced procollagen synthesis during the same time period raising the possibility that reduced procollagen synthesis is mediated, at least in part, by the observed alterations in the TGFB2-TGFBRII-Smad pathway (Quan TH et al, J Invest Dermatol, 2002, 119(2): 499-506). Accordingly, solar ultraviolet irradiation reduces collagen in photo-aged human skin by down-regulating the TGF-beta type II receptor (Quan T et al, Am J Pathol, 2004, 165(3): 741-51), and oxidative stress also impairs the TGF beta pathway via reduction of the TGFB type II receptor (He T et al, Age (Dordr), 2014, doi: 10.1007/s11357-014-9623-6). Notably, in contrast to TGFB2, TGFB1 and TGFB3 expression increases upon UV exposure (Quan TH et al, vide supra). In turn, the loss of TGF-beta type II receptor prevents downstream activation of Smad2/3 by TGF-beta, thereby, reducing expression of type I procollagen (He T et al, vide supra).

The present inventors have demonstrated that TGFBRII expression was clearly suppressed with UV irradiation and re-established with IK34720 (SEQ ID NO 3; IK) at 5h post UV treatment in primary fibroblasts (FERATONIC-p5).

Accordingly, there is provided polypeptides and compositions(s) as described herein for increasing procollagen synthesis and/or restoring UV-induced suppression of TGFβRII.

### Aging

Net skin aging is the result of two simultaneously occurring processes. The first, which can be referred to as “innate” or “intrinsic” aging affects the skin slowly inducing a partly-reversible degeneration of the connective tissues. The second process, which can be referred to as “extrinsic aging” or “photoaging” is mainly due to ultraviolet radiation which significantly contributes to a premature aging of the skin. The independence of these two pathways is exemplified by animal models that demonstrate that even in the absence of exposure to UV-radiation, skin still ages over time. The process of dermal aging is complex and involves myriad intracellular changes, and changes to the ECM, which result in a loss of, inter alia, skin elasticity, tone and pigment. Two of the primary causes of skin aging are accumulated cellular damage and destruction of the ECM of the skin.

UV-irradiation induces damage to the epidermis and dermis, which can result in long-term effects like photoaging. Photoaged skin displays alterations in the cellular component and extracellular matrix with an increase in, and accumulation of, disorganized elastin and fibrillin in the dermis. Further, photoaging can result in a loss of interstitial collagens which are the major structural proteins of the dermal connective tissue.

Accordingly, there is provided polypeptides and compositions as described herein for treating or preventing skin aging. The invention further provides a method for treating and/or preventing skin aging in a mammal, comprising administering (preferably topically) to the mammal an effective amount of a polypeptide or composition as described herein. In some embodiments, the aging is intrinsic aging. In some embodiments, the aging is extrinsic aging. In some embodiments, the extrinsic aging is photoaging.

The present invention provides polypeptides or compositions as described herein for inhibiting damage of cellular or extracellular components of the skin. In some embodiments, the damage is oxidative damage, such as damage caused by ROS (as discussed above), which can result in photoaging of the skin. In some embodiments, the damage is induced by UV-irradiation.

In some embodiments, the damage is enzymatic breakdown of the ECM. Accordingly, the present invention provides polypeptides and compositions as described herein for reducing degradation of the ECM and/or photoaging of the skin, in particular degradation following exposure of cells to UV-radiation. Further provided is a method for inhibiting degradation of the ECM and/or photoaging of the skin, comprising treating the skin, or a cell within the skin, with a polypeptide or composition as described herein.

As used herein, “skin aging” means the appearance of aging of the skin of a mammal. This includes both intrinsic aging and extrinsic aging, which is primarily caused by UV exposure of the skin. Therefore, “treating and/or preventing” aging or “anti-aging” refers to a slowing or inhibition in, and/or reversing of, the appearance of skin aging.

The underlying agents which induce these changes are reactive oxygen species (ROS), primarily UV-generated ROS. These ROS deplete and damage both non-enzymatic and enzymatic antioxidant defence systems of the skin. Consequently, in the presence of a depleted ROS defence system, antioxidants cause cellular damage primarily to cellular membranes, lipids and structural proteins such as elastin and collagens. Further underlying agents include Matrix metalloproteinases (MMPs).

### MMPs

Matrix metalloproteinases (MMPs) are zinc-containing endopeptidases which are able to degrade various components of extracellular matrix (ECM) proteins, such as collagen, fibronectin, elastin, and proteoglycans. UV-irradiation has been shown to increase the expression and secretion of MMPs in skin, which via their degradation of ECM can contribute to photoaging.

The present inventors demonstrated that that polypeptides in accordance with the present invention, such as the sequence RSKAKNPLYRRRRRRRRR (SEQ ID NO: 3), can reduce the activation of cellular matrix metalloproteinase 1 (MMP1).

Accordingly, the present invention provides polypeptides and compositions as described herein for reducing the concentration of MMPs following exposure of cells to UV-radiation. Further provided is a method for inhibiting MMP activity in a cell or tissue, comprising treating the cell or tissue (preferably topically) with a polypeptide or composition as described herein. In some embodiments, the MMP is MMP1.

By reducing the activity of MMPs in the skin, degradation of the skin's ECM, and photo-aging of the skin, can be reduced.

### Measures of Aging

Signs of aging include, but are not limited to, spider veins on the nose, cheeks and neck; pigmented spots, such as freckles, solar lentigines (known as age or liver spots), and uneven skin colour; general loss of skin tone; increase in number of wrinkles; increase in depth of wrinkles; increase in length of wrinkles; increase in lines such as forehead frown lines; the presence of benign actinic keratosis. As such, the assessment of these dermal features can be used to assess aging.

Without wishing to be bound by theory, in some embodiments, the polypeptides and methods of the present invention could be used to prevent or reduce the formation of one of the signs of aging provided above. In some embodiments, the polypeptides, compositions and methods of the present invention prevent or reduce the formation of spider veins on the nose, cheeks and neck. In some embodiments, the polypeptides, compositions and methods of the present invention prevent or reduce the formation of pigmented spots. In some embodiments, the polypeptides, compositions and methods of the present invention prevent or reduce the formation of freckles. In some embodiments, the polypeptides, compositions and methods of the present invention prevent or reduce the formation of solar lentigines. In some embodiments, the polypeptides, compositions and methods of the present invention prevent or reduce the formation of uneven skin colour. In some embodiments, the polypeptides, compositions and methods of the present invention prevent or reduce the formation of general loss of skin tone. In some embodiments, the polypeptides, compositions and methods of the present invention prevent or reduce the number of wrinkles. In some embodiments, the polypeptides, compositions and methods of the present invention prevent an increase in, or reduce the depth of, wrinkles. In some embodiments, the polypeptides, compositions and methods of the present invention prevent or reduce an increase in the length of wrinkles. In some embodiments, the polypeptides, compositions and methods of the present invention prevent or reduce an increase in forehead frown lines. In some embodiments, the polypeptides, compositions and methods of the present invention prevent or reduce the formation of benign actinic keratosis.

Further, methods are known in the art for assessing skin aging for different skin-types. For example R Bazin, E Doublet. Skin Aging Atlas. Volume 1. Caucasian Type. Med'Com; 2007; R Bazin and F Flament 2010 Skin Aging Atlas. Volume 2. Asian Type. Editions Med'Com, France; R Bazin, F Flament and F Giron 2012 Skin Aging Atlas. Volume 3. African-American Type. Editions Med'Com, France; R Bazin, F Flament and V Rubert, 2015, Skin Aging Atlas. Volume 4: Asian Type, Editions Med'Com, France; R Bazin, F Flament and H Qiu, 2017, Skin Aging Atlas. Volume 5: Photo-aging Face & Body Editions Med'Com, France.

The present disclosure is further described by the following examples. It is to be understood that the following description is for the purpose of describing particular embodiments only, and is not intended to be limiting with respect to the above description.

### EXAMPLE 1

ThepPolypeptides RSKAKNPLYRRRRRRRRR and rrrrrrrrrylpnkaksr promote wound healing.

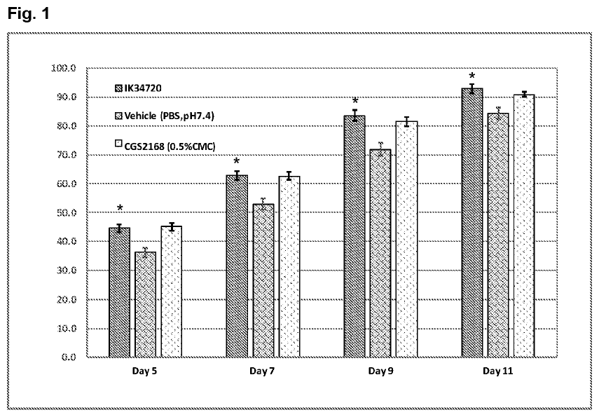

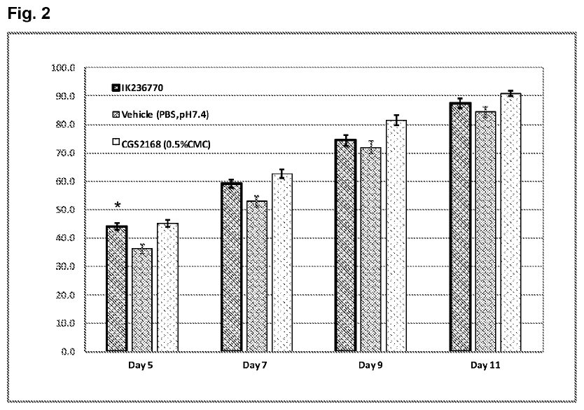

FIGS. 1 and 2 illustrate the effect of polypeptides IK34720 (SEQ ID NO: 3—RSKAKNPLYRRRRRRRRR) (FIG. 1) and the dextro-reverso sequence IK236770 (SEQ ID NO: 4—rrrrrrrrrylpnkaksr) (FIG. 2), on wound healing over 10 days in a murine wound model in accordance with the following protocol.

Three groups of 8 male ICR mice with a mean weight of 22 g (±2 g) were used in the present study. The animals were housed in individual cages during the study. Under isoflurane gas anaesthesia, the shoulder and back region of each animal was shaved and a sharp punch (ID 12 mm) was used to induce a wound by removing the skin, including panniculus carnosus and adherent tissues on Day 1.

Following induction of the wound the polypeptides IK34720 and IK236770 were topically administered to the site of the wound at a dosage of 10 μg per animal. Further a positive control group was administered 10 μg per animal of an alpha 2A agonist (CGS-21680) which has been shown to improve wound healing (Montesinos MC et al, J. Exp. Med., 1997, 186: 1615-1620). An additional group which comprised the administration of Phosphate buffered Saline (PBS) at pH 7.4 was used as a negative control.

The wound healing was analysed on Days 5, 7, 9 and 11 by tracing the periphery of the wound onto clear plastic sheets with the wound area calculated by an Image analyzer ProPlus (Media Cybernetics, Version 4.5.0.29. The percentage closure of the wound (%) was calculated. Percentage closure of the wound was determined on 5, 7, 9 and 11. ANOVA and Dunnett's test were applied to test significant significance between the treated groups (peptide and positive control groups) and the negative control group at each time point of measurement. Differences were considered statistically significant at P<0.05, and are indicated by *.

As shown in FIG. 1, topical application of 10 ug per mouse of IK34720 (SEQ ID NO: 3—RSKAKNPLYRRRRRRRRR) daily for 10 consecutive days was associated with a statistically significant increase (P<0.05) in wound closure percentage from Day 5 to Day 11. Further, and as expected, topical application of adenosine A2a agonist CGS-21680 (the positive control) at 10 μg/mouse was associated with an increase wound healing compared to the negative control, during the same study period.

As shown in FIG. 2, topical application of 10 ug per mouse of the polypeptide IK236770 (SEQ ID NO: 4) daily for 10 consecutive day was associated with a statistically significant increase in wound healing (as measured by wound closure % percentage) on day 5 compared with the negative control.

All values in FIGS. 1 and 2 represent mean±SEM at the designated time points.

This data illustrates that the polypeptides IK34720 (SEQ ID NO: 3—RSKAKNPLYRRRRRRRRR) and the dextro-reverso sequence IK236770 (SEQ ID NO: 4—rrrrrrrrrylpnkaksr) promotes wound healing when applied topically, compared to untreated controls.

### EXAMPLE 2

The polypeptide RSKAKNPLYRRRRRRRRR inhibits pCREB formation in primary keratinocytes when applied following keratinocyte exposure to solar simulated UV-irradiation, and restores UV-induced suppression of TGFβ receptor (TGFβRII).

CREB is phosphorylated at serine at position 133 (Ser-133) in response to cell exposure to UV-irradiation. Increases in CREB phosphorylation are associated with a range of functions including cell proliferation, cell cycle, metabolism, DNA repair, differentiation, inflammation, angiogenesis, immune responses and cell survival. Consequently, aberrant CREB phosphorylation is associated with tumour development, malignancy and survival (Steven. A and Seliger B., Oncotarget. 2016 Jun. 7; 7(23): 35454-35465).

The influence of the polypeptide IK34720 (SEQ ID NO: 3) on CREB phosphorylation was assessed using the following protocol.

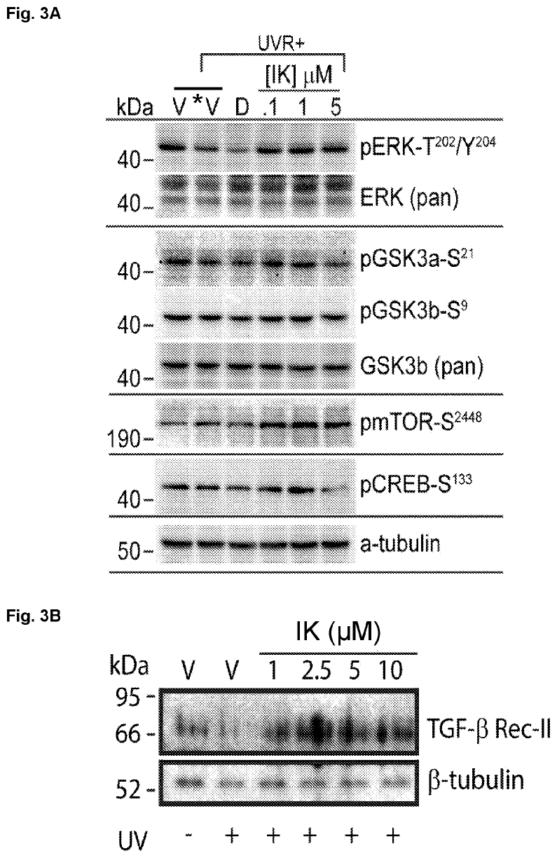

Keratinocytes were harvested and cultured as previously described (Gupta et al., J Invest Dermatol, 2007, 127(3):707-215). Keratinocytes, (passages 1-5) from 3 independent donors were used in the experiments illustrated in FIG. 3.

Positive control treatment (denoted “D” in FIG. 3) was prepared by solubilizing Calcitriol (1,25-dihydroxyvitamin D3 (1,25(OH)₂D₃)—Cayman Chemical, Mich., USA) in 100% spectroscopic ethanol (Merck, Darmstadt, Germany) and the final concentration was determined by spectroscopy (NanoDrop 2000, Thermo Fisher Scientific, Mass., USA). The polypeptide IK34720 (SEQ ID NO: 3) was solubilised in phosphate buffered saline (PBS) to a concentration of 1 mM at pH 7.2. A vehicle (denoted as “V” in FIG. 3) constituting 0.1% (v/v) spectroscopic ethanol and 0.1% (v/v) PBS was prepared for use as a negative control.

Cultured cells were irradiated by solar simulated UV-irradiation (ssUV) using an Oriel 1000W xenon-arc lamp (Stratford, Conn., USA). Irradiation was administered at an energy level of 400 mJ/cm² for UVB and 3600 mJ/cm² for UVA (total 4000 mJ/cm²), measured by an OL754 radiometer (Optronics Laboratories Inc., Orlando, Fla.). This dosage equates to approximately 4 min irradiation of the sun at 12 noon in October in Sydney, Australia. Non-irradiated control cells, (SHAM), were simultaneously processed with irradiated cells but protected from ssUV. Irradiation solution was PBS containing 5 mM D-glucose. Keratinocytes were plated in 6-well plates.

Immediately following irradiation 1 nM of 1,25(OH)₂D₃, negative control (vehicle) or 0.1 μM, 1 μM and 5 μM of IK34720 (“IK” in FIG. 3, SEQ ID NO: 3) were added to the cell cultures. Cells were subsequently lysed and western blot (as previously described in Rybchyn et al., vide supra, 2017, Rybchyn et al., JBC, 2011:286(27):23771-9) was used to analyse phosphorylation of ERK1/2-T202/Y204, GSK3α/β-S21/9, mTOR-S2448, CREB-S133 and a-tubulin (loading control). All antibodies used in the western blot were from Cell Signalling Technology (Mass., USA).

FIG. 3A illustrates the effect of the polypeptide IK34720 (SEQ ID NO: 3) on preventing UV-irradiation induced phosphorylation of Cyclic-AMP-responsive element-binding protein (CREB). As can be seen from FIG. 3, the polypeptide IK34720 (SEQ ID NO: 3) inhibits UV-induced phosphorylation of serine at position 133 of CREB (CREB S-133) in primary keratinocytes when applied following keratinocyte exposure to solar simulated UV-irradiation.

Both intrinsic skin aging and UV-induced skin aging lead to a reduction of TGFβRII required for type 1 procollagen synthesis. Furthermore, stimulation of procollagen synthesis by sunscreen protection is linked to TGFβ signalling.

The influence of the polypeptide IK34720 (SEQ ID NO: 3) on TGFβRII phosphorylation was assessed using the following protocol.

Human skin dermal fibroblasts were cultured from keratome biopsies of healthy adult human skin. Cells were between passages three and six. For UV irradiation, subconfluent cells were washed once with phosphate-buffered saline (PBS) and irradiated with UV. Immediately following irradiation, negative control (vehicle) or 1 μM, 2.5 μM, 5 μM and 10 μM of IK34720 (“1K” in FIG. 3B, SEQ ID NO: 3) were added to the cell cultures.

For Western analysis, nuclear extracts were prepared, membrane fractions were prepared and resolved on 8% SDS-polyacrylamide gel electrophoresis, transferred to polyvinylidene difluoride membrane, and reacted with primary antibodies. Blots were visualized and quantified.

FIG. 3B illustrates the effect of the polypeptide IK34720 (SEQ ID NO: 3) on restoring UV-induced suppression of TGFβ receptor (TGFβRII)). As can be seen from FIG. 3B, UV treatment supresses TGFβRII levels, and the polypeptide IK34720 (SEQ ID NO: 3) restored TGFβRII expression in UV treated cells. TGFBRII expression was clearly suppressed with UV irradiation and re-established with IK34720 (SEQ ID NO 3; IK) at 5 h post UV treatment in primary fibroblasts (FERATONIC-p5).

This indicates the polypeptide RSKAKNPLYRRRRRRRRR, when applied following UV-irradiation, restores UV-induced suppression of TGFβRII.

### EXAMPLE 3

The polypeptide RSKAKNPLYRRRRRRRRR increases ATP levels in primary keratinocytes exposed to solar simulated UV-irradiation.

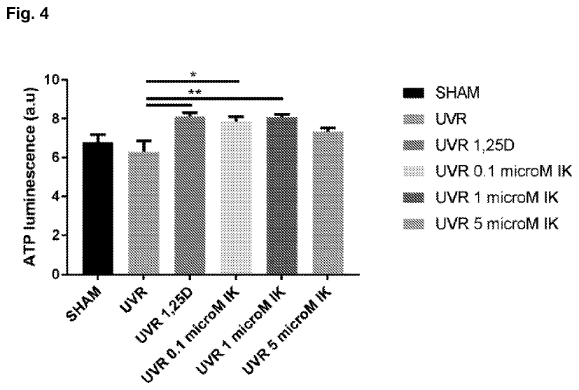

FIG. 4 illustrates the effect of the polypeptide IK34720 (SEQ ID NO: 3) on ATP levels in primary keratinocytes exposed to solar simulated UV-irradiation. Inadequately repaired DNA damage following UV exposure has been demonstrated to be a causative factor in skin cancer development. DNA repair requires energy, but after UV exposure skin cells have a limited capacity to produce ATP, which is the primary source of the energy.

The influence of the polypeptide IK34720 (SEQ ID NO: 3) on intracellular ATP following UV exposure was assessed using the following protocol.

Primary keratinocytes were harvested as described above in Example 2 and were either exposed to ssUV-irradiation (UVR), as described above in Example 2 or not irradiated (SHAM), in the presence or absence of Calcitriol (positive control) or polypeptide IK34720 (SEQ ID NO: 3; RSKAKNPLYRRRRRRRRR), at the concentrations described in Example 2. Calcitriol (1α, 25-dihydroxyvitamin D3), has been demonstrated to reduce UV-induced DNA damage and photocarcinogenesis in a variety of models.

ATP levels were determined 1.5 hours after UVR using the CellTiter-Glo® 2.0 Assay (Promega, Wis., USA) as described previously (Rybchyn et al., vide supra, 2017). All data in FIG. 4 is shown as mean±SD. *p<0.05, **p<0.01. Cells were prepared and irradiated as described above.

As can be seen from FIG. 4, the polypeptide IK34720 (“IK”, SEQ ID NO: 3) enhanced the ATP levels in primary keratinocytes following exposure to solar simulated UV, to a similar level as Calcitriol. Increases in ATP levels are proposed to assist in the repair of DNA following UV-induced damage.

This indicates the polypeptide RSKAKNPLYRRRRRRRRR, when applied following UV-irradiation, increases ATP levels following exposure to solar simulated UV.

### EXAMPLE 4

The polypeptide RSKAKNPLYRRRRRRRRR, when applied topically to skin following UV-irradiation, decreases oxidative stress in the skin.

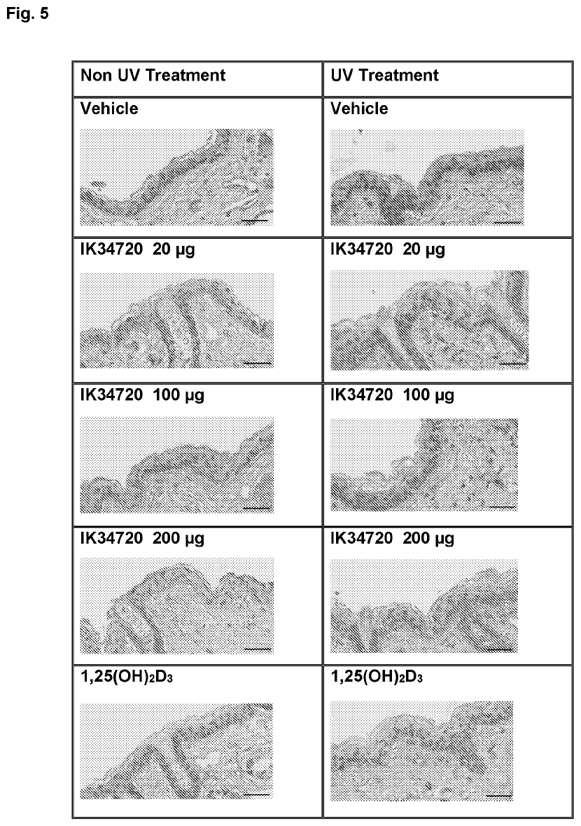

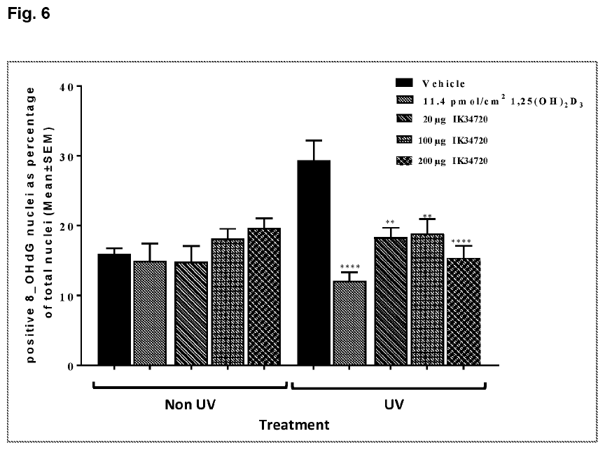

FIGS. 5 to 6 illustrate the effect of the polypeptide IK34720 (SEQ ID NO: 3) on inhibiting UV-induced oxidative stress in the skin.

8-hydroxy-2′-deoxyguanosine or 8-oxo-7,8-dihydro-2′-deoxyguanosine (8-OHdG/8-oxodG) is one of the predominant forms of free radical-induced in oxidative lesions with the concentrations of 8-OHdG within a cell being a measurement of oxidative stress. The role of the polypeptide IK34720 (SEQ ID NO: 3) in UV-induced oxidative stress was assessed using the following protocol.

Female Skh:hr1 hairless mice were exposed to solar simulated UV-irradiation (UVR) by exposure to one fluorescent UVB tube (Philips TL40W 12R/S, Eindhoven, The Netherlands) in combination with 6 UVA tubes (Hitachi 40W F40T 10/BL, Tokyo, Japan). The UVA and UVB irradiation was filtered through a 0.125 mm cellulose acetate sheet (Grafix Plastics, Cleveland, Ohio) as previously described (Dixon KM et al., Biochem Mol Biol, 2007, 103(3-5):451-6). UV-irradiated mice were subjected to a single exposure equal to three times the minimal erythemal dose (MED) of UVR (3.98 kJ/m2 UVB and 63.8 kJ/m2 UVA). Irradiated animals were randomly allocated to 5 treatment groups (n=3 per group).

Immediately following irradiation, mice in each treatment group for assessing oxidative stress were treated topically on the dorsal surface with either vehicle (base lotion containing ethanol, propylene glycol and water to a final solvent ratio of 2:1:1 respectively), 11.4 pmol/cm2 of Calcitriol (“1,25(OH)₂D₃), or a treatment of 20 μg, 100 μg or 200 μg the polypeptide IK34720 (SEQ ID NO: 3) in 100 uL of aqueous solution.

Biopsies were taken from UV-irradiated dorsal skin 3 h post-UVR and fixed in Histochoice fixative (Amresco, Solon Ohio) for 6 h. Skin samples were paraffin-embedded and 5 micrometre sections were cut for all analyses. Sections were subjected to routine hematoxylin and eosin staining for visualization of sunburn cells at 40× magnification, and the number of sunburn cells per linear millimetre of skin section recorded.

Levels of 8-oxo-2′-deoxyguanosine (8oxodG) indicative of oxidative stress were detected by immunohistochemistry and image analysis (Dixon KM et al., Cancer Prey Res 2011, 4(9):1485-94) as set out below.

Slides were deparaffinised and rehydrated in a series of graded ethanol solutions. Antigen retrieval was performed using Proteinase K at 37° C. for 30 minutes, followed by treatment of sections with 2 N HCI (in 70% ethanol) for 15 minutes and subsequently 50 mM Tris buffer for a further 15 minutes. For 8oxodG, slides were further treated with RNase A at 200 μg/mL (Amresco, Ohio, USA) at 37° C. for 30 minutes. Subsequent steps were carried out using the Dako Animal Research Kit using the method prescribed by the manufacturer (Dako, Glostrup, Denmark). The anti-thymine dimer antibody (Sigma-Aldrich, Mo., USA) was used at 10 μg/mL to indicated DNA damage in the form of CPDs, while the 8oxodG antibody (Trevigen, Md., USA) was used at 2.5 μg/mL to visualise oxidative stress.

As can be seen in the immunohistology presented in FIG. 5, and quantified in FIG. 6, the polypeptide IK34720 (SEQ ID NO: 3) inhibits 8-OHdG formation in the skin of Skh:hr1 mice following acute UV-irradiation. In FIG. 5, dark staining in nuclei indicates the presence of 8-oxo-dG.

The quantification of the immunohistological data for 8oxodG demonstrated that there was a statistically significant decrease in all treatment groups exposed to UV-irradiation compared to the vehicle (negative) control (**p<0.01, ****p<0.0001). No significant staining was observed with a monoclonal mouse IgG isotype used as an isotype staining control (data not shown).

This indicates the polypeptide RSKAKNPLYRRRRRRRRR, when applied topically to skin following UV-irradiation, decreases oxidative stress in skin cells induced by UV-irradiation.

### EXAMPLE 5

The polypeptide RSKAKNPLYRRRRRRRRR, when applied topically to skin following UV-irradiation, decreases DNA damage.

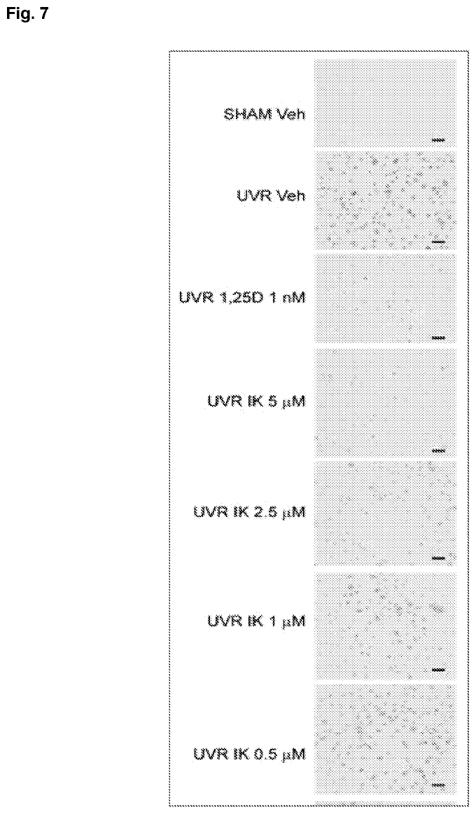

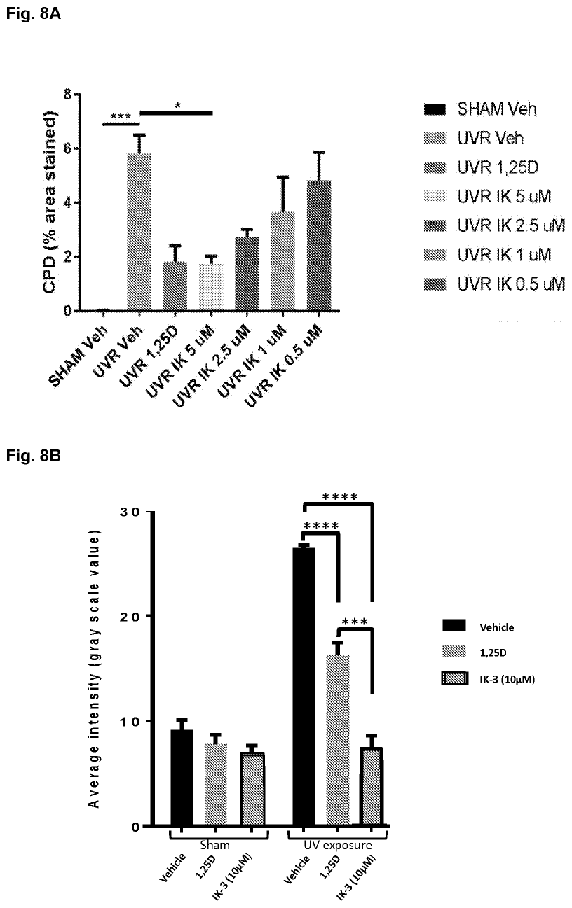

FIGS. 7 and 8 illustrate the effect of the polypeptide IK34720 (SEQ ID NO: 3; “IK”) on UV-induced DNA damage, as demonstrated by formation of Cyclobutane pyrimidine dimers (CPDs) in vitro.

Exposure to UV-radiation triggers a cascade of chemical reactions, and many molecular products (photolesions) are formed that can inhibit polymerases, cause misreading during transcription or replication of DNA, or lead to arrest of replication. Cyclobutane pyrimidine dimers (CPD) are a common product of UV exposure and their concentration can quantify DNA damage.

An in vitro assay was conducted allowing for densitometry analysis of CPD detection by IHC in keratinocytes 3 h following UV-irradiation in accordance with the following protocol.

Keratinocytes were harvested and cultured as described above at Example 2. Samples were split into two groups, being UV irradiated (UVR) and non-UV irradiated (SHAM). UV-irradiation was performed by solar simulated UV-irradiation for cells, also as described above at Example 2. Irradiation solution was PBS containing 5 mM D-glucose.

Following irradiation, keratinocytes were treated with either Calcitriol, vehicle control (Veh) or the polypeptide 1K34720 (SEQ ID NO: 3; “1K”) at concentrations of 0.5 μM, 1 μM, 2.5 μM or 5 μM.

Harvested keratinocytes were plated on poly-D-lysine coated coverslips in 96 well plates and irradiated (or sham treated) as described above in at Example 2 in PBS containing 5 mM D-glucose. Keratinocytes were fixed 3 h after UV-irradiation. Immunohistochemistry and image analysis were performed as described previously (Gordon-Thomson et al., Photochem Photobiol Sci, 2012, 11(12):1837-47; Gupta et al., vide supra, Rybchyn MS et al., J Invest Dermatol, 2018;138(5):1146-1156). Thymine dimers were detected using the anti-thymine dimer antibody (Sigma-Aldrich, Mo., USA) at 10 μg/mL, and quantified as an index of CPD, as previously described (Douki T and Cadet J, Biochemistry,2001, 40(8):2495-501).

As shown in FIGS. 7 and 8, the polypeptide IK34720 (SEQ ID NO: 3—“IK”, respectively) decreases CPD levels in primary keratinocytes exposed to solar simulated UV-irradiation in a dose dependent manner. Keratinocytes treated with 5 μM of the polypeptide IK34720 (SEQ ID NO: 3) had a comparable reduction in CPD levels to keratinocytes treated with Calcitriol, and had a statistically significant (*p<0.05) reduction in CPD levels compared to vehicle-treated UV-irradiated keratinocytes.

This data demonstrates the polypeptide IK34720 (SEQ ID NO: 3; “IK”) reduces DNA damage in primary keratinocytes following exposure to UV-irradiation.

In FIG. 8B, CPD was calculated in terms of average intensity of CPD staining. Importantly, FIG. 8B demonstrates that at 10 uM, the polypeptide IK34720 (SEQ ID NO: 3; “IK”) is more effective at inhibiting CPD formation than the positive control 1,25 D.

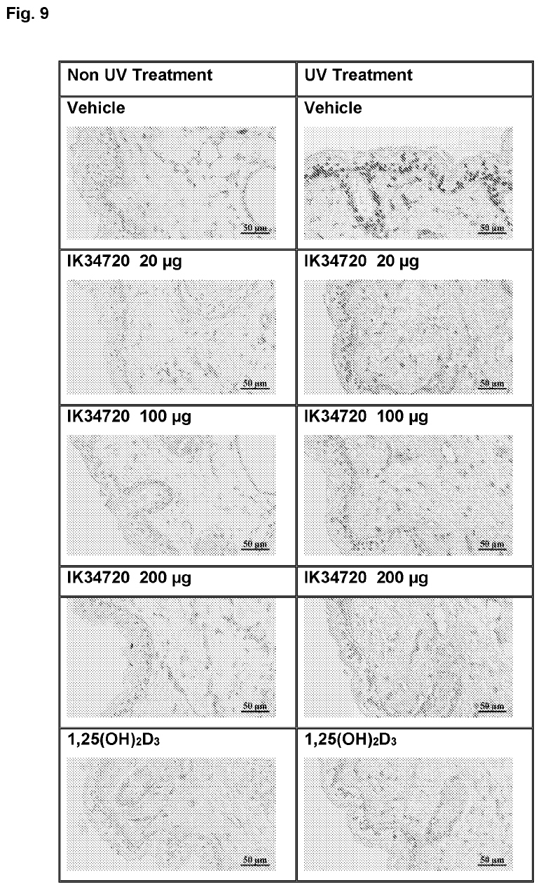

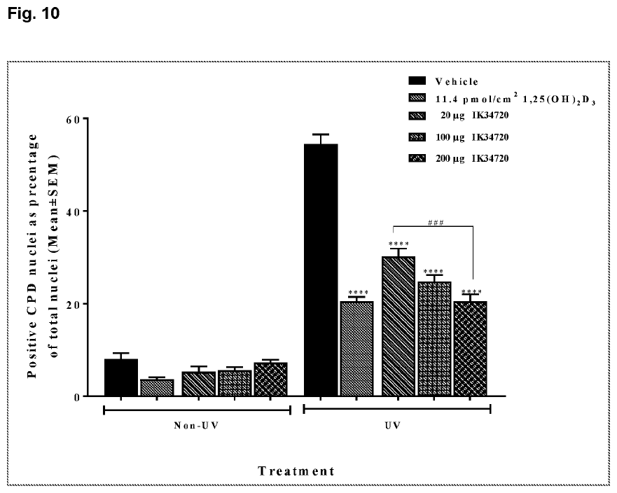

FIGS. 9 and 10 illustrate the effect of the polypeptide IK34720 (SEQ ID NO: 3; “IK”) on UV-induced DNA damage, as demonstrated by formation of Cyclobutane pyrimidine dimers (CPDs) in vitro, in accordance with the following protocol.

Female Skh:hr1 hairless mice were exposed to solar simulated UV-irradiation as described above at Example 4. Immediately after irradiation mice were treated topically on the dorsal surface also as described in Example 4. Subsequently, skin was fixed at 3 hours post-UVR, and subjected to immuno-histochemical staining using the antibody directed against thymine dimers (CPD) described above at 10 μg/ml. No significant staining was observed with a monoclonal mouse IgG isotype control (not shown). In FIG. 9, dark staining in nuclei indicates the presence of thymine dimers (CPD), and the % area stained is presented. Immunohistology was quantified as per Example 4.

As can be seen in the immunohistology presented in FIG. 9A, and quantified in FIG. 10, the polypeptide IK34720 (SEQ ID NO: 3) inhibits CPD formation in the skin of Skh:hr1 mice following acute UV-irradiation.

The quantification of the immunohistological data for CPD nuclei demonstrated that there was a statistically significant decrease in all treatment groups exposed to UV-irradiation compared to the vehicle (negative) control (**p<0.01, ****p<0.0001). No significant staining was observed with a monoclonal mouse IgG isotype used as an isotype staining control (data not shown).

This indicates the polypeptide RSKAKNPLYRRRRRRRRR, when applied topically to skin following UV-irradiation, decreases DNA damage, as measured by the formation of CPD nuclei in skin cells induced by UV-irradiation.

### EXAMPLE 6

The polypeptide RSKAKNPLYRRRRRRRRR, when applied topically to skin following UV-irradiation, reduces the number of apoptotic cells.

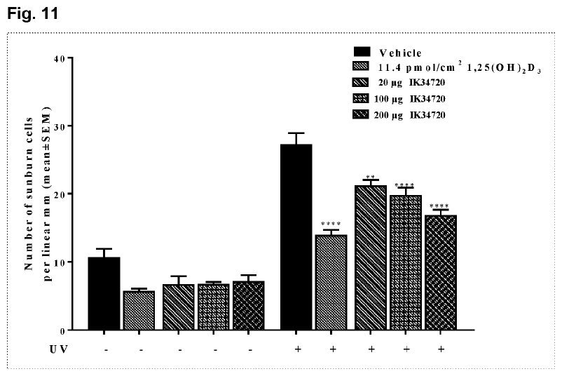

FIG. 11 illustrates the effects of the polypeptide IK34720 (SEQ ID NO: 3) on cell apoptosis following UV-irradiation.

UV-induced DNA damage in the form of elevated CPDs can induce mutations in epidermal cells leading to the development of cancer cells. Reduction of CPDs through application of DNA repair enzymes prevents the risk of UV-induced skin cancer and reduce the level of apoptotic cells following UV-induced damage. The number of apoptotic skin cells following UV-irradiation was assessed in accordance with the following protocol.

Female Skh:hr1 hairless mice were prepared, irradiated and biopsied according to the methods of Example 4. Prior to biopsy, and immediately after irradiation, mice were treated with either a base lotion vehicle (negative control), 11.4 pmol/cm2 of Calcitriol in vehicle (positive control) the polypeptide IK34720 (SEQ ID NO: 3) in water, at 20 ug, 100 ug or 200 ug in a total volume of 100 uL.

Biopsies were taken from UV-irradiated dorsal skin 3 hrs post-UVR and fixed in Histochoice fixative (Amresco, Solon OH) for 6 h. Skin samples were paraffin-embedded and 5 micrometre sections were cut for all analyses. Sections (24 h after UV) were subjected to routine hematoxylin and eosin staining for visualization of sunburn cells. The stained sections were examined under a Zeiss-Axioplan light microscope at 40× magnification, and the number of sunburn cells per linear millimetre of skin section recorded.

As shown in FIG. 11, the topical application of the polypeptide IK34720 (SEQ ID NO: 3) reduced the number of apoptotic keratinocytes following UV-irradiation.

This data demonstrates the polypeptide IK34720 (SEQ ID NO: 3) reduces keratinocyte apoptosis following exposure to UV-irradiation.

### EXAMPLE 7

The polypeptide RSKAKNPLYRRRRRRRRR, when applied topically to human skin following UV-irradiation damage, decreases DNA damage, and reduces the number of apoptotic sunburn cells.

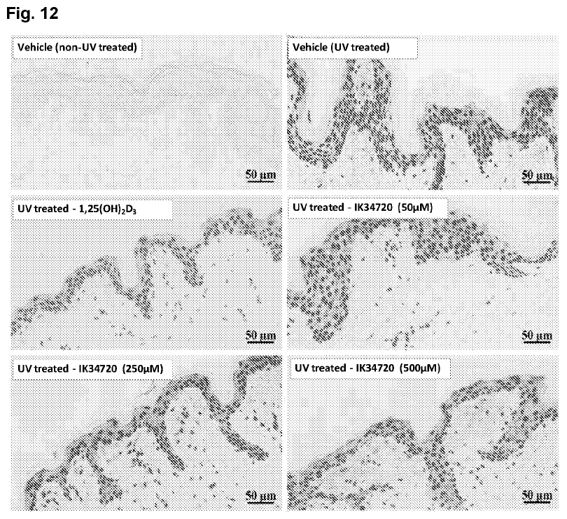

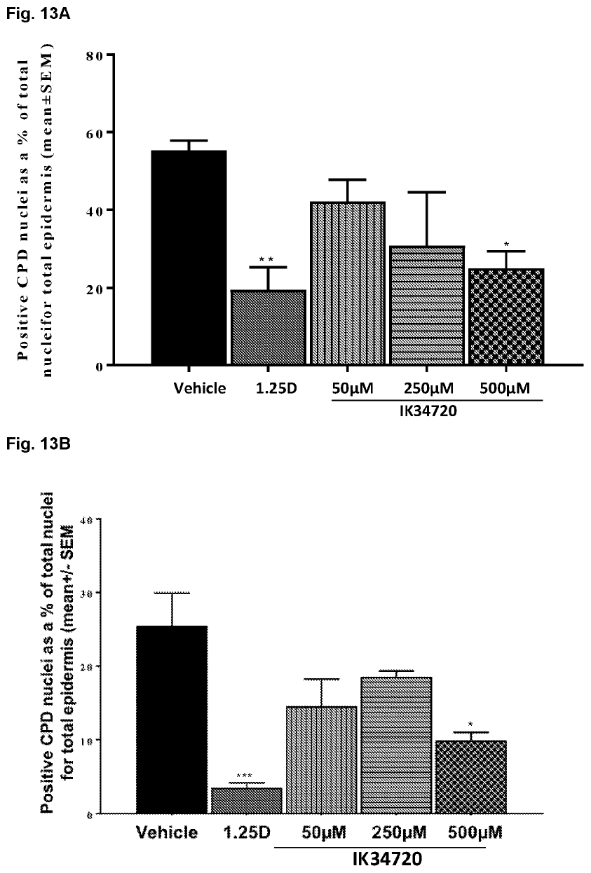

FIGS. 12 and 13 illustrate the effect of the polypeptide IK34720 (SEQ ID NO: 3) on UV-induced DNA damage, as demonstrated by formation of CPDs in human explants, in accordance with the following protocol.

Human skin removed at elective surgery was cleaned, dissected and cut into pieces of approx. 5 mm×5 mm. Each piece was placed in a well of a 96 well plate, with individual plates prepared to assess the effects 3 hrs post treatment. All samples were performed in triplicate for each treatment. Skin was subjected to UV-radiation from solar-simulated UV source for 1.5 h (UVR), or non-irradiation (SHAM). Given that human skin is thicker than mouse skin, human skin explants were exposed to a radiation dose three times higher than that used for mouse skin, i.e., 20 J/cm2 versus 7 J/cm2, respectively, and the output spectrum of the laboratory solar simulator has previously been reported (Rybchyn MS et al, J Invest Dermatol. 2018 May; 138(5):1146-1156) and treated immediately after with a vehicle control, Calcitriol (1 nM) or the polypeptide IK34720 (SEQ ID NO: 3) at concentrations of 50 μM, 250 μM or 500 uM.

Samples harvested at 3 hrs were fixed, section, stained and quantified as described in Example 5.

As can be seen in the immunohistology presented in FIG. 12, UV-irradiation increases the number of cells having CPDs (illustrated as dark spots) in all irradiated group 3 hrs post irradiation/treatment, with samples treated with Calcitrol or varying concentrations of the polypeptide IK34720 (SEQ ID NO: 3) having fewer stained cells. This data is quantified in FIG. 13A and B for two individuals where the percentage of CPD positive cells was statistically significantly reduced when treated with 500 μM of the polypeptide IK34720 (SEQ ID NO: 3).

This indicates the polypeptide RSKAKNPLYRRRRRRRRR, when applied topically to skin following UV-irradiation, can reduce the formation of CPD in human skin explants following acute UV-irradiation at 3 hours post irradiation. This also confirms the mouse results presented in FIGS. 9 and 10.

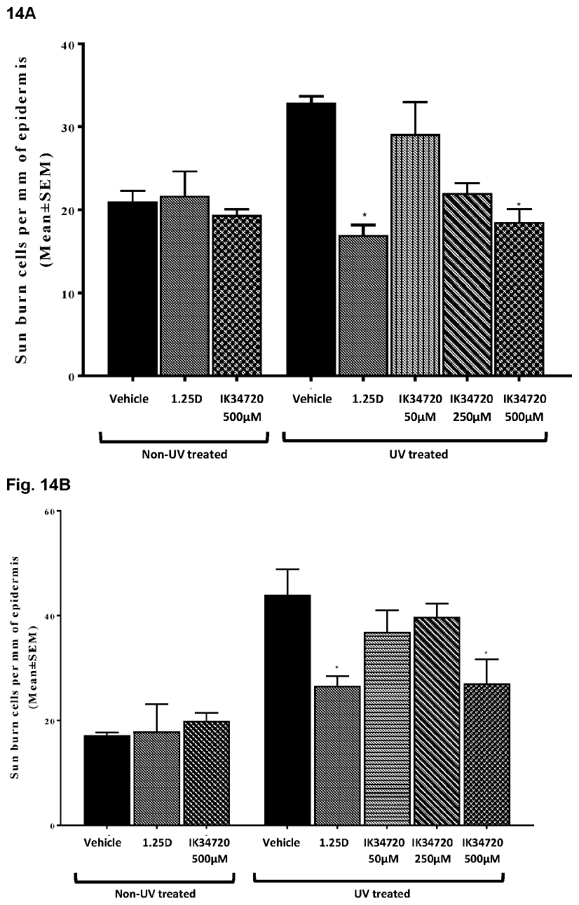

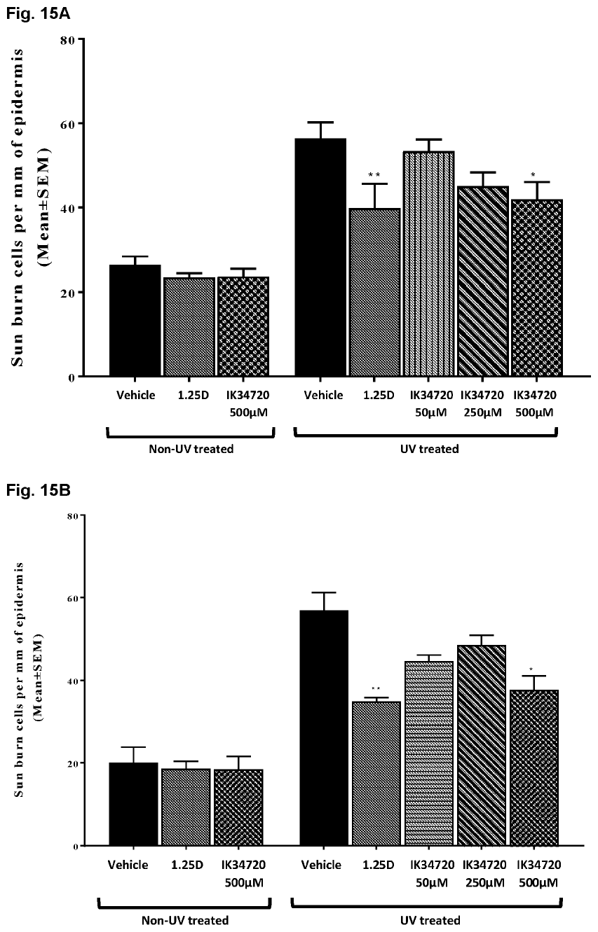

FIGS. 14 and 15 illustrate the effect of the polypeptide IK34720 (SEQ ID NO: 3) on the number of apoptotic cells in human skin explants.

Skin explants were cultured irradiated and treated as described above for CPD, and subsequent to treatment were stained and quantified as described for Example 6.

FIG. 14 shows the number sunburn cells per mm of epidermis of human skin explants 3 hours post UV-irradiation in the various treatment groups. FIG. 15 shows the number sunburn cells per mm of epidermis of human skin explants 24 hours post UV-irradiation in the various treatment groups.

As shown in FIGS. 14 and 15, treatment with 500 μMof the polypeptide IK34720 (SEQ ID NO: 3) statistically significantly reduces the number of apoptotic cells 3 hrs and 24 hrs post UV-irradiation.

This data indicates that the polypeptide IK34720 (SEQ ID NO: 3) reduces the number of apoptotic sunburn cells when applied topically to skin following UV-irradiation.

### EXAMPLE 8

The polypeptide RSKAKNPLYRRRRRRRRR inhibits Matrix Metalloproteinase-1 (MMP-1) activity in vitro and in human skin explants.

UV-induced skin damage initiates elevated MMP-1 in human skin cells that can lead to destruction of collagen, which is a hallmark of photoaging. It is known that the major enzyme responsible for collagen I digestion, matrix metalloproteinase 1 (MMP-1) is induced by exposure to sunlight (Dong KK et al, Exp Dermatol, 2008, 17(12): 1037-44).

The effect of the polypeptide IK34720 (SEQ ID NO: 3) on MMP-1 activity was evaluated using the following protocol.

Human rheumatoid synovial fibroblast MMP-1 proenzyme, activated with APMA for 60 minutes at 37° C., was used as an active enzyme. The polypeptide IK34720 (SEQ ID NO: 3) at a concentration of 5 μM. 10 μM 800 μM and 1000 μM was pre-incubated with 8 nM activated enzyme in modified MOPS buffer pH 7.2 for 60 minutes at 37° C. The reaction was initiated by addition of 4 mM Mca-Pro-Leu-Gly-Leu-Dap-Ala-Arg followed by a 2 hour incubation period. Determination of the amount of Mca-Pro-Leu-Gly formed was read spectrofluorimetrically at 340 nm/400 nm.

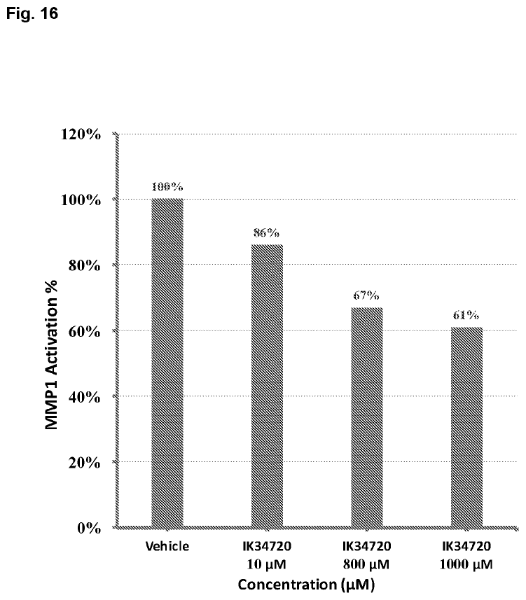

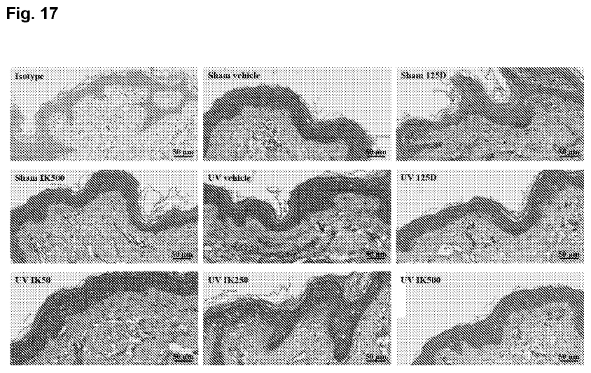

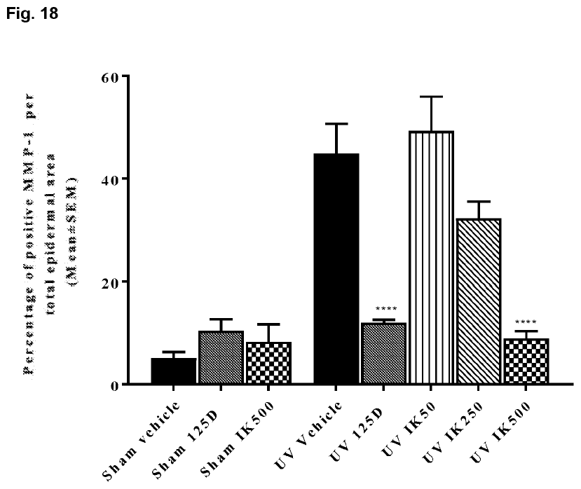

FIG. 16 illustrates the effect of the polypeptide IK34720 (SEQ ID NO: 3) on the activity of MMP-1 in vitro, and FIGS. 17 and 18 illustrate the effect of the polypeptide IK34720 (SEQ ID NO: 3) in human skin explants following UV-induced skin damage.

As can be seen in FIG. 16, the polypeptide IK34720 (SEQ ID NO: 3) inhibits MMP-1 activation in a dose dependent manner. This data indicates that the polypeptide IK34720 (SEQ ID NO: 3) can be used to reduce photoaging over time.

The effect of the polypeptide IK34720 (SEQ ID NO: 3) on MMP-1 activity in human skin was evaluated using the following protocol.

FIGS. 17 and 18 illustrate the effect of the polypeptide IK34720 (SEQ ID NO: 3) on MMP1 expression, in accordance with the following protocol.

Human skin (2× human explant tissues) removed at elective surgery was cleaned, dissected and cut into pieces of approx. 5mm×5mm. Each piece was placed in a well of a 96 well plate, with individual plates prepared to assess the effects 3 hrs post treatment. Skin was subjected to UV-radiation from solar-simulated UV source for 1.5 h (UVR), or non-irradiation (SHAM), and treated immediately after with a vehicle control, Calcitriol (1 nM) or the polypeptide IK34720 (SEQ ID NO: 3) at concentrations of 50 μM, 250 μM or 500 uM. In FIG. 17, IK50, IK250 and IK500 as μM concentrations refers to 15 μg, 75 μg and 150 μg of IK34720 per 120 μL culture medium/explant, respectively.

Samples harvested at 3 hrs were fixed, sectioned, immunohistochemically stained for MMP1 and quantified for MMP1 expression, by expressing epidermal area stained for MMP1 as a function of total epidermal area examined.

As can be seen in the immunohistology presented in FIG. 17, UV-irradiation increases the number of cells staining for MMP-1 activity (illustrated as dark spots) in all irradiated groups 3 hrs post irradiation/treatment, with samples treated with Calcitrol or varying concentrations of the polypeptide IK34720 (SEQ ID NO: 3) having fewer stained cells. No significant staining was observed with a monoclonal mouse IgG isotype used as an isotype staining control (data not shown).

This data is quantified in FIG. 18 for the explants where the percentage of MMP1 positive cells is statistically significantly reduced when treated with 500 μM of the polypeptide IK34720 (SEQ ID NO: 3). This indicates the polypeptide RSKAKNPLYRRRRRRRRR, when applied topically to skin following UV-irradiation, decreases MMP1 expression induced by UV-irradiation.

### EXAMPLE 9

The polypeptide RSKAKNPLYRRRRRRRRR inhibits UV-induced lipid peroxidation in dermal skin fibroblasts cell lysates.

Polyunsaturated fatty acids (lipids) are frequently used in cosmetics as soaps, skin care lotions and creams, after-shaves, make-up removers, etc. because in contrast to saturated lipids they help to maintain the skin's natural oil barrier. Moreover, the less saturated a lipid is the higher is the liquidity of the lipid. However, unsaturated lipids are more prone to oxidation from agents such as UV-irradiation and hence anti-oxidants, e.g., vitamins, are often added to skin care formulations.

Oxidising agents can alter lipid structure, creating lipid peroxides that result in the formation of malondialdehyde which can be measured as thiobarbituric acid-reactive substances (TBARS).

As shown in Example 4, the polypeptide RSKAKNPLYRRRRRRRRR when applied topically to skin following UV-irradiation decreases UV-induced oxidative stress in the skin.

The ability of the polypeptide RSKAKNPLYRRRRRRRRR to alter UV-induced oxidative damage of cell-derived lipids was examined using the TBARS thiobarbituric acid-reactive substances (TBARS) lipid peroxidation assay.

After irradiation or sham-irradiation of dermal fibroblasts, supernatants (900 μL) were collected. In brief, 90 μL of butylated hydroxytoluene (2% w/w in ethanol) was added and samples kept frozen (−20° C.) until the TBARS assay. Petri dishes were washed twice with 1 mL of HBSS, cells scraped in 600 μL of water and 60 μL of 0.5% aqueous Triton X100 added to this solution, for protein determination carried out by the Lowry method (Lowry OH et al, J. Biol. Chem., 1951, 193:265-275). TBARS were fluorometrically assayed as described (Morlière P et al, Biochim. Biophys. Acta, 1991, 1084:261-268). Briefly, thawing samples were heated in the presence of thiobarbituric acid in acidic conditions, and TBARS extracted with 1-butanol and fluorometrically quantified (λexc=515 nm and λem=550 nm). Tetraethoxypropane, which quantitatively yields the malondialdehyde-thiobarbituric acid adduct in the assay conditions, was used for calibration. TBARS values, expressed in malondialdehyde (MDA) equivalents, were normalized to the cell protein for each Petri dish. Each TBARS determination was performed in triplicate (e.g., with three Petri dishes per data point).

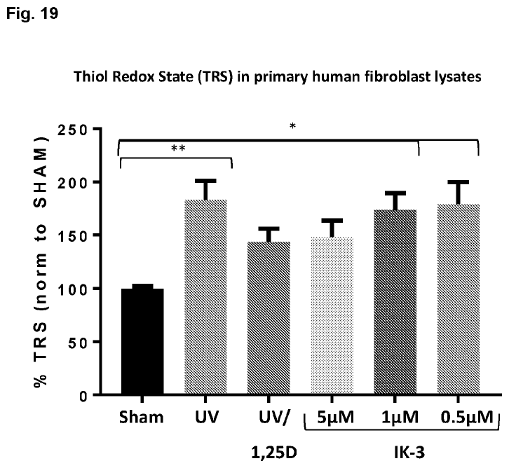

As can be seen in FIG. 19, UV-irradiation increases the number of TBA reactive substances (TRS) in all irradiated groups post irradiation/treatment. The significant difference in TRS between Sham and UV irradiation in the absence of treatment is not seen in the presence of either 1,25 D or RSKAKNPLYRRRRRRRRR (“IK-3”) at the highest concentration (5 μM) compared with lower doses of RSKAKNPLYRRRRRRRRR.

This indicates the polypeptide RSKAKNPLYRRRRRRRRR, decreases UV-induced oxidative damage of cell-derived lipids in dermal skin fibroblast cell lysates.

### EXAMPLE 10

Effect of IK14800 on melanin production.

Melanoma induction by UVA requires melanin pigment and UVB irradiation initiates melanoma. A desirable effect of skin care products is to reduce skin pigmentation that occurs from sunlight exposure.

EXAMPLE 2 shows the polypeptide RSKAKNPLYRRRRRRRRR inhibits pCREB formation in primary keratinocytes when applied following keratinocyte exposure to solar simulated UV-irradiation. Phosphorylated CREB induces activation of the microphthalmia (MITF) transcription factor, which is a myc-like master transcription factor that, in melanocytes, drives expression of tyrosinase and other pigment biosynthetic enzymes.

The ability of the polypeptide RSKAKNPLYRRRRRRRRR to alter melanin production was examined.

In brief, MM1418-C1 lightly pigmented melanoma cells were grown in 24 well culture plates and treated with 10 μM RSKAKNPLYRRRRRRRRR (“IK-3”) for 24 hours prior to being exposed to 2 kJ/m2 UVB radiation. Controls (not treated with RSKAKNPLYRRRRRRRRR) labelled “Con” and “UVB”, respectively, were exposed to either 0 or 2 kJ/m2 UVB radiation. Cell groups were returned to the incubator for 24 hours and then the levels of melanin and protein in the cells were measured spectrophotometrically. In each experiment triplicate samples were measured. Data shown in FIG. 19 indicate the average values±SEM (μg melanin/mg cell protein) exposed to 0 or 2 kJ/m2 UVB which had been pre-treated with 0 or 10 μM of RSKAKNPLYRRRRRRRRR 24 hours prior to being exposed to UVB radiation. The t-test (2-tailed) was used to determine statistical significance, *p<0.05, ***p<0.001.

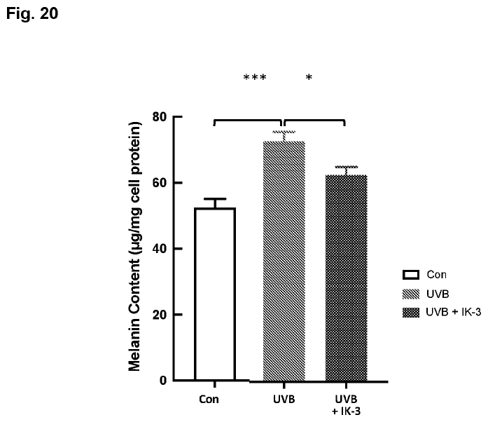

As can be seen in FIG. 20, UVB-irradiation significantly increases the melanin content of cells. Importantly, cells treated with RSKAKNPLYRRRRRRRRR prior to UVB treatment show significantly decreased melanin content, relative to cells not treated with RSKAKNPLYRRRRRRRRR.

This indicates the polypeptide RSKAKNPLYRRRRRRRRR, decreases melanin induction by UVB.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

The subject headings used herein are included only for the ease of reference of the reader and should not be used to limit the subject matter found throughout the disclosure or the claims. The subject headings should not be used in construing the scope of the claims or the claim limitations.

Also, it is to be noted that, as used herein, the singular forms “a”, “an” and “the” include plural aspects unless the context already dictates otherwise. As such, the terms “a” (or “an”), “one or more,” and “at least one” are used interchangeably herein.

1-53. (canceled)

54. A method for promoting wound healing, or treating and/or preventing a skin condition in a mammal, comprising administering to the mammal an effective amount of a polypeptide comprising an isolated or purified polypeptide comprising the amino acid sequence RSKAKNPLYR (SEQ ID NO: 1), or the dextro-reverso form of the amino acid sequence (rylpnkaksr; SEQ ID NO: 2), and a polyarginine amino acid sequence region

55. The method according to claim 54, wherein the isolated or purified polypeptide comprises the amino acid sequence RSKAKNPLYRRRRRRRRR (SEQ ID NO: 3), or rrrrrrrrrlpnkaksr (SEQ ID NO: 4).

56. The method according to claim 54, wherein the isolated or purified polypeptide consists of the amino acid sequence RSKAKNPLYRRRRRRRRR (SEQ ID NO: 3), or rrrrrrrrrlpnkaksr (SEQ ID NO: 4).

57. The method according to claim 54, wherein the skin condition is oxidative damage.

58. The method according to claim 54, wherein the skin condition is DNA damage.

59. The method according to claim 54, wherein the skin condition is damage induced by sunlight exposure.

60. The method according to claim 54, wherein the skin condition is ultraviolet radiation induced damage.

61. The method according to claim 60, wherein the ultraviolet radiation induced damage is UVB induced damage or UVA induced damage.

62. The method according to claim 54, wherein the skin condition is characterised by CPD and/or 8-OHdG formation.

63. The method according to claim 54, wherein the skin condition is skin aging and/or skin wrinkling.

64. A method for inhibiting matrix metalloproteinase 1 (MMP1) expression and/or MMP1 activity in a cell, comprising treating the cell with an effective amount of a polypeptide comprising an isolated or purified polypeptide comprising the amino acid sequence RSKAKNPLYR (SEQ ID NO: 1), or the dextro-reverso form of the amino acid sequence (rylpnkaksr; SEQ ID NO: 2), and a polyarginine amino acid sequence region.

65. The method according to claim 64, wherein the isolated or purified polypeptide comprises the amino acid sequence RSKAKNPLYRRRRRRRRR (SEQ ID NO: 3), or rrrrrrrrrlpnkaksr (SEQ ID NO: 4).

66. The method according to claim 64, wherein the isolated or purified polypeptide consists of the amino acid sequence RSKAKNPLYRRRRRRRRR (SEQ ID NO: 3), or rrrrrrrrrlpnkaksr (SEQ ID NO: 4).

67. A method for treating and/or preventing damage to the extracellular matrix of the skin, comprising administering to the skin an effective amount of a polypeptide comprising an isolated or purified polypeptide comprising the amino acid sequence RSKAKNPLYR (SEQ ID NO: 1), or the dextro-reverso form of the amino acid sequence (rylpnkaksr; SEQ ID NO: 2), and a polyarginine amino acid sequence region.

68. The method according to claim 67, wherein the isolated or purified polypeptide comprises the amino acid sequence RSKAKNPLYRRRRRRRRR (SEQ ID NO: 3), or rrrrrrrrrlpnkaksr (SEQ ID NO: 4).

69. The method according to claim 67, wherein the isolated or purified polypeptide consists of the amino acid sequence RSKAKNPLYRRRRRRRRR (SEQ ID NO: 3), or rrrrrrrrrlpnkaksr (SEQ ID NO: 4).

70. A method for promoting wound healing, or treating and/or preventing a skin condition in a mammal, comprising administering to the mammal, to the wound, or to a site of the skin condition, an effective amount of a polypeptide comprising an isolated or purified polypeptide comprising the amino acid sequence RSKAKNPLYR (SEQ ID NO: 1), or the dextro-reverso form of the amino acid sequence (rylpnkaksr; SEQ ID NO: 2), and a polyarginine amino acid sequence region.

71. A method for inhibiting CREB phosphorylation in a cell, comprising treating the cell with an effective amount of a polypeptide comprising an isolated or purified polypeptide comprising the amino acid sequence RSKAKNPLYR (SEQ ID NO: 1), or the dextro-reverso form of the amino acid sequence (rylpnkaksr; SEQ ID NO: 2), and a polyarginine amino acid sequence region.

72. A method for inhibiting 8-OHdG formation in a cell, comprising treating the cell with an effective amount of a polypeptide comprising an isolated or purified polypeptide comprising the amino acid sequence RSKAKNPLYR (SEQ ID NO: 1), or the dextro-reverso form of the amino acid sequence (rylpnkaksr; SEQ ID NO: 2), and a polyarginine amino acid sequence region.

73. A method for inhibiting CPD formation in a cell, comprising treating the cell with an effective amount of a polypeptide comprising an isolated or purified polypeptide comprising the amino acid sequence RSKAKNPLYR (SEQ ID NO: 1), or the dextro-reverso form of the amino acid sequence (rylpnkaksr; SEQ ID NO: 2), and a polyarginine amino acid sequence region.


SEQUENCE LISTING (4 sequences)

SEQ ID NO: 1 - protein, 10 aa, Artificial Sequence
  Synthetic polypeptide
RSKAKNPLYR

SEQ ID NO: 2 - protein, 10 aa, Artificial Sequence
  Synthetic polypeptide
  SITE (1)..(10): Dextro reverso
RYLPNKAKSR

SEQ ID NO: 3 - protein, 18 aa, Artificial Sequence
  Synthetic polypeptide
RSKAKNPLYRRRRRRRRR

SEQ ID NO: 4 - protein, 18 aa, Artificial Sequence
  Synthetic polypeptide
  SITE (1)..(18): Dextro reverso
RRRRRRRRRYLPNKAKSR